(19) United States
(12) Patent Application Publication
(10) Pub. No.: US 2002/0147429 A1
(43) Pub. Date: Oct. 10, 2002

(54) Syringes, connectors, and syringe and connector systems for use in fluid delivery systems

(76) Inventors: Kevin P. Cowan, Allison Park, PA (US); Mark W. Hitchins, Sewickley, PA (US); Ralph H. Schriver, Tarentum, PA (US); Frederick W. Trombley III, Gibsonia, PA (US); Joseph E. Bisegna, Cheswick, PA (US); Herbert M. Grubic, Pittsburgh, PA (US); Francis J. Sciulli, Crafton, PA (US); David M. Reilly, Glenshaw, PA (US); Arthur E. Uber III, Pittsburgh, PA (US); Frank B. McFall, Allison Park, PA (US)

Correspondence Address: GREGORY L BRADLEY, MEDRAD INC, ONE MEDRAD DRIVE, INDIANOLA, PA, 15051

(21) Appl. No.: 10/079,066

(22) Filed: Feb. 20, 2002

Related U.S. Application Data
(60) Provisional application No. 60/302,322, filed on Jun. 29, 2001.
(60) Provisional application No. 60/270,310, filed on Feb. 20, 2001.

(51) Int. Cl. A61M 5/00

(52) U.S. Cl. 604/187; 604/191

(57) ABSTRACT

A syringe for use in a medical injection procedure in connection with connectors having a tapered fitting includes a syringe tip having a cooperating tapered fitting including a passage therein in fluid communication with an interior of the syringe. The syringe also includes a first connecting member having a radially inward threaded portion on an inner surface thereof adapted to connect to a first type of connector including a tapered fitting having at least one radially outward extending flange adapted to connect to the radially inward threaded portion of the first connecting member. In that regard, the first connecting member allows connection of the syringe to, for example, a standard luer fitting connector. The syringe further includes a second connecting member having at least one attachment member such as a radially outward extending flange adapted to connect to a second type of connector which includes a tapered fitting and a cooperating attachment member to engage the at least one attachment member of the second connecting member. The second connecting member allows connection of the syringe to improved tapered fitting connectors described herein.

### CROSS-REFERENCE TO RELATED APPLICATIONS

The present application claims the benefit of U.S. Provisional Application Serial No. 60/302,322, filed on Jun. 29, 2001, and U.S. Provisional Application Serial No. 60/270,310, filed on Feb. 20, 2001, the disclosures of which are incorporated herein by reference.

### BACKGROUND OF THE INVENTION

The present invention relates to syringes, connectors, and syringe and connector systems for use in fluid delivery systems, and, especially, to syringes, connectors, and syringe and connector systems for use in medical fluid delivery systems in which one or more fluids are delivered to a patient.

In many medical procedures, such as drug delivery, it is desirable to inject a fluid into a patient. Likewise, numerous types of contrast media (often referred to simply as contrast) are injected into a patient for many diagnostic and therapeutic imaging procedures. In some medical procedures (for example, computed tomography (CT), angiography, and nuclear magnetic resonance/magnetic resonance imaging (MRI)) it is desirable to deliver a liquid such as contrast medium in a timed fashion under pressure. Relatively high pressures and timed boluses are typically achieved through the use of powered injectors.

In many such fluid delivery systems it is necessary to form a fluid connection between separate fluid path components. For example, it may be necessary to connect an injector-powered syringe to flexible plastic tubing that, in turn, is connected to a catheter inserted into a patient. A common connector used in the medical arts is the luer connector or luer lock. FIG. 1 illustrates a common design of luer connector 10. Luer connector 10 includes a male connector or member 20 and a female connector or member 30. Male member 20 includes a conduit 24 having a taper on an outside wall thereof of typically approximately 6%. Female member 30 includes a conduit or fitting 34 therein having a taper of approximately 6% to form a mating fit with the outside wall of conduit or fitting 24 of male member 20. Male member 20 and female member 30 are typically connected via radially inward projecting threading 40 attached to male member 20, which cooperates with radially outward extending flanges 36 and 38 on female luer member 30 to create a sealed connection between male member 20 and female member 30.

Many fluid connectors for use in medical procedures, including luer connectors, exhibit substantial drawbacks, not the least of which include fragility/breakability (for example, from over tightening) and difficulty in forming a connection. Medical personnel are under increasingly difficult time and physical constraints during various medical procedures. Quite often, many fluid path elements must be connected and/or disconnected in a relatively short time under stressed and/or emergency conditions.

It is thus very desirable to develop syringes, connectors, and syringe and connector systems that are durable, connect and disconnect simply and quickly, and yet provide a reliable fluid path connection.

### SUMMARY OF THE INVENTION

In general, the present invention provides syringes, connectors, adapters, systems and methods of connection for use in medical fluid delivery systems in which many of the problems associated with prior fluid path connections are eliminated. The connectors of the present invention are suitable for use both at low pressures and at the relatively high pressures used in powered injection procedure (for example, pressures of 300 psi and above). In general, the connectors of the present invention can be connected using only a single hand. With many of the connectors of the present invention, rotation is not required to form an adequate seal. In those connectors of the present invention in which rotation is desirable, such rotation is generally no more than approximately ½ turn (or 180°), and more preferably, no more than approximately ¼ turn (or 90°). In several embodiments, the connectors of the present invention prevent over tightening and the damage associated therewith. Moreover, the connectors of the present invention can be fabricated to be generally open or non-enclosing so that the fluid path in the vicinity of the connectors is readily visible to the operator.

In one aspect, the present invention provides a syringe for use in a medical injection procedure in connection with a connector having a tapered fitting (for example a luer fitting). The syringe includes a syringe tip including a cooperating tapered fitting having a passage therein in fluid communication with an interior of the syringe. The syringe also includes a first connecting member including a radially inward threaded portion on an inner surface thereof adapted to connect to a first type of connector. The first type of connector includes a tapered fitting and has at least one radially outward extending flange to connect to the radially inward threaded portion of the first connecting member. For example, the first type of connector can be a standard luer connector. The syringe further includes a second connecting member including at least one abutment member to connect to a second type of connector. The second type of connector includes a cooperating tapered fitting and a cooperating attachment member including at least one radially inward projecting flange to engage the at least one abutment member of the second connecting member.

The second connecting member can, for example, include one or more radially outward extending flanges. The flange can be a threaded flange. The second connecting member can alternatively include at least one radially inward extending channel.

In one embodiment, the first connecting member is positioned on a radial interior side of a generally cylindrical member surrounding the cooperating tapered fitting and the second connecting member is positioned on a radial exterior side of the generally cylindrical member. The cooperating tapered fitting can, for example, be a male luer fitting.

In another aspect, the present invention provides a syringe and connector system for use in a medical injection procedure. The syringe and connector system includes a syringe as generally described above and a connector including a cooperating tapered fitting and a cooperating attachment member. The cooperating attachment member preferably includes at least one radially inward projecting flange to engage the attachment member (for example, an abutment member) of the first connecting member. The connector also includes at least one port in fluid connection with the cooperating tapered fitting.

The abutment member of the syringe can, for example, include a single radially outward extending flange oriented generally perpendicular to an axis of the syringe tip. The abutment member can also include a single radially inward extending channel oriented generally perpendicular to an axis of the syringe tip. The abutment member can also include a radially outward extending threaded flange (or threading).

In one embodiment in which the abatement member includes radially outward extending threading, the radially inward projecting flange of the connector can include a threaded portion to engage the threading of the attachment member. The threaded portion of the connector can be rotatable relative to the cooperating tapered fitting. Such relative rotation of the threaded portion of the connector can facilitate connection and prevent rotation/twisting of fluid path elements (for example, tubing) attached to a port of the connector.

The cooperating attachment member of the connector can, for example, include at least one extending arm comprising at least one radially inward extending flange. Preferably, the cooperating attachment member of the connector includes a plurality of extending arms, wherein each of the arms includes at least one radially inward extending flange. The radially inward extending flanges of the arms can be biased radially inward when the connector and the syringe are engaged. In several embodiments, the arms are resilient, flexing arms and are biased radially inward by a bending moment. The arms can also be biased radially inward by a locking member attached to the connector (for example, by a collet or collar slidably disposed around the extending arms).

In several connectors of the present invention, an axial compressive force is maintained between the cooperating fittings when the connector is connected. For example, the connector can further include a biasing member to provide an axially oriented force directed to retain the tapered fitting of the syringe and the cooperating tapered fitting of the connector in sealing engagement. The biasing member can, for example, include at least one resilient spring arm in operative connection with the cooperating tapered fitting. In several embodiments, the biasing member includes a plurality of resilient spring arms in operative connection with the cooperating tapered fitting. The spring arms of the present invention can, for example, deform elastically and/or permanently to create a compressive axial force between cooperating fittings.

In the case that the attachment member of the syringe includes a threaded flange or threading, the radially inward extending flanges of the extending arms can include at least one groove formed thereon to mate with the threading of the attachment member. The connector can, for example, be rotatable relative to the syringe after connection thereto to provide (additional) axially oriented force directed to retain the tapered fitting of the syringe and the cooperating tapered fitting of the connector in sealing engagement. The arms can prevent over tightening during rotation of the connector by flexing radial outward during rotation to slip over the threading of the attachment member. Preferably, no more than 90° of rotation is required to provide a desired level of axial force.

In one embodiment, the cooperating attachment member of the connector includes at least one radially inward extending flange having an adjustable angle of orientation. The flange has a locking orientation in which it resists disengagement from the abutment member and a disengagement orientation in which the flange can pass over the abutment member of the syringe to be placed in engagement with or to be removed from engagement with the abutment member of the syringe. The locking orientation can, for example, form a first angle with respect to the axis of the syringe tip, while the disengagement orientation forms a second angle with respect to the axis of the syringe tip. In general, the second angle is closer to perpendicular to the axis of the syringe tip than the first angle. In this embodiment, the abutment member can, for example, be a generally cylindrical surface. For example, no flange or channel is required to cooperate with the flange of the connector.

In another aspect, the present invention provides a connector for use with a tapered fitting assembly having at least one attachment member. The connector includes a cooperating tapered fitting, at least one port in fluid connection with the cooperating tapered fitting, and a cooperating attachment member attached to the cooperating fitting to engage the attachment member of the tapered fitting assembly. In general, a predetermined level of force is preferably required to cause the cooperating attaching elements to form a cooperating connection with the at least one attachment member of the tapered fitting assembly.

The cooperating attachment member can engage the attachment member via relative axial motion of the cooperating tapered fitting and the fitting assembly. The cooperating attachment member can also engage the attachment member via relative axial motion of the cooperating attachment member and the fitting assembly followed by rotation of the cooperating attachment member relative to the fitting assembly. Preferably, however, the cooperating attachment is rotated no more than 90° during connection.

As described above, the cooperating attachment member can include one or a plurality of axially extending arms having at least one radially inward extending flange projecting therefrom.

As also described above, the connector can also include a biasing member or mechanism to provide axially oriented force directed to retain the tapered fitting assembly and the cooperating tapered fitting of the connector in sealing engagement.

In another aspect, the present invention provides a connector for use with a tapered fitting assembly including a cooperating tapered fitting, a cooperating attachment member in operative attachment with the cooperating fitting to engage the tapered fitting assembly, and at least one port in fluid connection with the cooperating tapered fitting. The cooperating attachment member includes a radially inwardly extending flange having an adjustable angle of orientation. As described above, the flange has a locking orientation in which it resists disengagement from the tapered fitting assembly and a disengagement orientation in which the flange can be placed in engagement with the tapered fitting assembly or be removed from engagement with the tapered fitting assembly.

In a further aspect, the present invention provides a connector for use with a tapered fitting assembly having at least one attachment member. The connector includes a cooperating tapered fitting and a plurality of resilient, extending arms in operative attachment with the cooperating tapered fitting. Each of the arms includes at least one radially inward extending flange as described above. A predetermined level of force is required to cause the cooperating radially inward extending flanges to form a cooperating connection with the at least one attachment member of the tapered fitting assembly.

A rearward surface of the radially inward extending flanges of the arms of this embodiment and other embodiments of the present invention can be sloped forward to cause the arms to flex radially outward when the connector is moved to contact the flanges with the attachment member of the tapered fitting assembly (for example, a radially outward extending flange).

The connectors of the present invention can include a retainer to attach a length of tubing thereto by contacting the exterior wall of the tubing. Other fluid path elements can be attached to the ports of the connector of the present invention. For example, the connectors can include a valve (for example, a check valve) connected to the port.

In another aspect, the present invention provides an injector system for use in a medical injection procedure. The injector system includes a powered injector including a drive member, a syringe including an engagement mechanism for removable attachment of the syringe to the injector and a plunger adapted to cooperate with the drive member of the injector. A syringe tip on a forward end of the syringe includes a tapered fitting having a passage therein in fluid communication with an interior of the syringe. The syringe further includes a first connecting member having at least one attachment member. The injector system also includes a connector including a cooperating tapered fitting, a cooperating attachment member to engage the attachment member of the first connecting member, and at least one port in fluid connection with the cooperating tapered fitting. A predetermined level of force is require to cause the cooperating attaching member to form a connection with the at least one attachment member of the first connecting member.

In a further aspect, the present invention provides a connector for use with a tapered fitting assembly having at least one attachment member. The connector includes a cooperating tapered fitting, a cooperating attachment member operable to engage the attachment member of the tapered fitting assembly and at least one port in fluid connection with the cooperating tapered fitting. The cooperating attachment member includes a plurality of extending arms. Each of the arms includes at least one radially inward extending attaching element. The connector further includes a biasing member to provide axially oriented force directed to retain the tapered fitting assembly and the cooperating tapered fitting of the connector in sealing engagement.

As described above, the biasing member can include at least one spring arm in operative connection with the cooperating tapered fitting. The connector can, for example, be formed from an integral piece of resilient polymeric material.

In still a further aspect, the present invention provides method for fabricating a connector for use with a tapered fitting assembly having at least one attachment member including the step of forming the connector from an integral piece of polymeric material. The connector can, for example, be formed to include a cooperating tapered fitting, a cooperating attachment member to engage the attachment member of the tapered fitting assembly, and at least one port in fluid connection with the cooperating tapered fitting. In one embodiment, the cooperating attachment member includes a plurality of extending arms as described above and a biasing member to provide axially oriented force directed to retain the tapered fitting assembly and the cooperating tapered fitting of the connector in sealing engagement.

In another aspect, the present invention provides a method of connecting a connector including a tapered fitting with a tapered fitting assembly including a cooperating tapered fitting. The method includes the steps of forming an axial compressive force directed to maintain connection between the tapered fitting of the connector and the cooperating tapered fitting of the tapered fitting assembly under pressure, and indicating to the user that the axial compressive force has been attained.

The method can further include the step of engaging radially inward extending flanges of extending, resilient arms of the connector with at least one abutment member of the tapered fitting assembly. The radially inward extending flanges can be biased radially inward to engage the abutment member of the tapered fitting assembly.

The present invention provides in another aspect an adapter for use in a medical injection procedure in connection with connectors having a tapered fitting. The adapter includes a forward portion including a male tapered fitting and a surface surrounding at least a portion of the male tapered fitting. The surface includes at least one attachment member to connect to a connector including a female tapered fitting and a cooperating attachment member to engage the at least one attachment member. The adapter also includes a rearward portion including a rearward fitting in fluid connection with the male tapered fitting.

The rearward fitting can be a female luer fitting. The surface can, for example, include a radially inward threaded portion on an inner surface thereof.

The male tapered fitting, the female tapered fitting and the surface of the adapter can formed integrally, for example, of a molded polymeric material.

The present invention provides in a further aspect, an adapter and connector system for use in a medical injection procedure including an adapter as described above and a connector including a female tapered fitting to engage the male tapered fitting of the adapter, a cooperating attachment member to engage the at least one attachment member of the adapter, and at least one port in fluid connection with the female tapered fitting.

In another aspect, the present invention provides a connector for use in a medical procedure to connect to a fitting assembly including an interference fitting. The connector includes a cooperating interference fitting and an indicator to inform the user that the interference fitting of the fitting assembly and the cooperating interference fitting of the connector have been brought together with an axial compressive force suitable to maintain sealed connection between the tapered fitting of the connector and the cooperating tapered fitting of the tapered fitting assembly under a known pressure.

The indicator can, for example, include an attachment member on the connector that comes into connection with a cooperating attachment member on the fitting assembly. The connection resists axial disconnection of the connector from the fitting assembly. The attachment member on the connector can, for example, include at least one axially extending, flexible arm including a radially inward projecting abutment member to connect to the cooperating attachment member as described above.

The interference fitting of the fitting assembly can be a tapered fitting and the cooperating interference fitting of the connector can be a cooperating tapered fitting. For example, the tapered fitting of the fitting assembly can a luer fitting and the tapered fitting of the connector can be a cooperating luer fitting. At least one of the interference fitting of the fitting assembly and the cooperating interference fitting of the connector can include a deformable surface to improve sealed connection therebetween. As described above, an axial compressive force can be maintained between the interference fitting of the fitting assembly and the cooperating interference fitting of the connector while connected.

In a further aspect, the present invention provides a connector for use in a medical procedure to connect to a fitting assembly including a fitting and an attachment member. The connector includes a cooperating fitting having an annular deformable seal, a port in fluid connection with the cooperating fitting, and a cooperating attachment member. The deformable seal (for example, an O-ring type seal) is preferably under positive engagement. The cooperating attachment member cooperates with the attachment member to maintain the connector in connection with the fitting assembly so that the deformable seal maintains a sealed connection with the fitting of the fitting assembly.

In one embodiment, the annular seal is in fluid contact with a fluid flowing through the connector. The force with which the annular seal contacts the fitting of the fitting assembly increases with increasing fluid pressure.

The present invention also provides in a further aspect a connector for connection to a male fitting assembly including a surface surrounding a male fitting. The connector includes a length of deformable conduit. The end of the deformable conduit includes a radially outward extending flange dimensioned to form a sealed connection with the surrounding surface. In one embodiment, the surrounding surface of the male fitting assembly includes radially inward extending threading. The flange of the deformable conduit can, for example, abut the threading to prevent disconnection of the connector from the male fitting assembly when under fluid pressure. The male fitting can, for example, be a male luer fitting.

In another aspect, the present invention provides a connector for connection to a male fitting assembly including a male fitting. The connector includes a length of deformable conduit and a slidable collet surrounding the conduit. The conduit is dimensioned to form a sealed connection between the male fitting and the deformable conduit when the deformable conduit is connected to the male fitting and the collet is slid thereover. In one embodiment, the male fitting assembly includes a generally cylindrical surface surrounding the male fitting, and the conduit is dimensioned to slide between the deformable conduit and generally cylindrical surface.

In still a further embodiment, the present invention provides a connector including a male fitting assembly having a male interference fitting and a generally cylindrical barrel surrounding the male fitting. The barrel includes one or more pitched or canted flanges positioned adjacent a generally axially oriented slot on the interior wall thereof. The flange(s) can, for example, follow the path of a helix. The connector also provides a female fitting assembly comprising a female interference fitting and at least one radially outward projection dimensioned to pass through the slot of the male fitting assembly and be rotated into abutment with one of the pitched flanges.

In one embodiment, the male fitting assembly comprises two generally axially oriented slots positioned generally opposite of each other. A first plurality of pitched flanges is positioned between the slots on one side of the interior of the barrel, and a second plurality of pitched flanges is positioned between the slots on another side of the interior of the barrel. The female fitting assembly can include two radially outward projections, which are dimensioned to pass through one of the slots of the male fitting assembly and be rotated into abutment with one of the pitched flanges. The female member can include a stop member to stop rotation of the female fitting assembly after a predetermine amount of rotation. Preferably, the rotation required to form a sealed connection between the female fitting and the male fitting is no more than 90°.

### BRIEF DESCRIPTION OF THE DRAWINGS

FIG. 1 illustrates a side, cross-sectional view of a standard luer connection.

FIG. 2A illustrates a side view of a dual syringe injector system attachable to a fluid path via standard luer connections.

FIG. 2B illustrates a side view of a dual syringe system attachable to a fluid path via standard luer connections including threaded swivel nuts on the syringes.

FIG. 3A illustrates a side view of an embodiment of a syringe and connector system of the present invention in which the connector is in an exploded or disconnected state.

FIG. 3B illustrates a side, cross-sectional view of the syringe and connector system of FIG. 3A in which the connector is connected to the syringe.

FIG. 3C illustrates an enlarged view of FIG. 3B.

FIG. 4A illustrates a side view of another embodiment of a syringe and connector system of the present invention in which the connector is disconnected from the syringe.

FIG. 4B illustrates an enlarged side view of the syringe tip and connector of FIG. 4A in which the connector is connected to the syringe tip.

FIG. 4C illustrates a perspective view of the syringe and connector system of FIG. 4A in which the connector is connected to the syringe.

FIG. 4D illustrates a side view of the connector of FIG. 4A disconnected from the syringe in which the conduit member is shown in cross-section and to which a valve mechanism and a tubing retainer have been added.

FIG. 5A illustrates a side view of another embodiment of a syringe and connector system of the present invention in which the connector is disconnected from the syringe.

FIG. 5B illustrates a side, cross-sectional view of the connector of FIG. 5A.

FIG. 5C illustrates a perspective view of the syringe and connector system of FIG. 5A in which the connector is connected to the syringe.

FIG. 5D illustrates side views of the connector of FIG. 5A in a non-stressed or non-flexed state and in a stressed or flexed state for connection to the syringe.

FIG. 5E illustrates a side view of the syringe and connector system of FIG. 5A in which the connector is connected to the syringe tip.

FIG. 6A illustrates a side, cross-sectional view of another embodiment of a syringe and connector system of the present invention in which the connector is disconnected from the syringe.

FIG. 6B illustrates a side, cross-sectional view of the syringe and connector system of FIG. 6A in which the connector is being brought into connection with the syringe.

FIG. 6C illustrates a perspective view of FIG. 6B.

FIG. 6D illustrates a perspective view of the connector of FIG. 6A in an exploded or disconnected state and a side, cross-sectional view of the locking member of the connector.

FIG. 6E illustrates a side, cross-sectional view of the syringe and connector system of FIG. 6A in which the connector and the syringe are connected.

FIG. 7A illustrates a side view of another embodiment of a syringe and connector system of the present invention in which the connector is disconnected from the syringe.

FIG. 7B illustrates a side view of the syringe and connector system of FIG. 7A in which the connector and the syringe are connected.

FIG. 7C illustrates a perspective view of the syringe and connector system of FIG. 7A in which the connector and the syringe are connected and the orientation of the attachment member is being changed to allow disconnection.

FIG. 7D illustrates a side view of the connector of FIG. 7A in which the orientation of the attachment member is being changed to a state in which the connector can be connected to or disconnected from the syringe.

FIG. 8A illustrates a front perspective view of an adapter and connector system of the present invention aligned for attachment to a syringe.

FIG. 8B illustrates a rear perspective view of the adapter of FIG. 8A.

FIG. 8C illustrates a front perspective view of the adapter of FIG. 8A.

FIG. 8D illustrates a side, cross-sectional view of the adapter of FIG. 8A.

FIG. 8E illustrates a front perspective view of another embodiment of an adapter of the present invention including a rear fitting for connection to a length of tubing.

FIG. 9A illustrates a side view of an embodiment of a fluid path connection of the present invention including a flange with a forward sloping surface and a rearward sloping surface.

FIG. 9B illustrates a side view of an embodiment of a fluid path connection including multiple flanges, in which each flange includes a forward sloping surface and a rearward sloping surface.

FIG. 9C illustrates a perspective view of an embodiment of a bayonet-type connector of the present invention.

FIG. 10 illustrates a side view of an embodiment of a connector of the present invention suitable for connection to a plurality of fittings, including male and/or female fittings.

FIG. 11A illustrates a side, cross-sectional view of another embodiment of a connector of the present invention in which a large portion of the connector is formed from an integral piece of elastomeric material.

FIG. 11B illustrates a top view of an embodiment of arced abutment members for use with the connector of FIG. 11A.

FIG. 11C illustrates a perspective view of the connector of FIG. 11A.

FIG. 12A illustrates a side view of another embodiment of a connector of the present invention aligned for connection to a syringe.

FIG. 12B illustrates a side view of the connector of FIG. 12A aligned for connection with a length of tubing.

FIG. 12C illustrates a side view of axial movement of the connector of FIG. 12A relative to a cooperating fitting to form a connection between the fitting of the connector and the cooperating fitting.

FIG. 12D illustrates a side view of axial displacement of extending or gripping arms of the connector of FIG. 12A to form a locking connection between the gripping arms and a channel formed in the cooperating fitting.

FIG. 12E illustrates a side view of compression of the forward ends of the gripping arms to release the gripping arms from connection with the channel formed in the cooperating fitting.

FIG. 12F illustrates a side view of the connector of FIG. 12A with a female fitting thereon.

FIG. 12G illustrates a side view of a connector similar in operation to the connector of FIG. 12F with a male fitting thereon.

FIG. 12H illustrates a side, cross-sectional view of an axially compressible seal positioned within a female fitting.

FIG. 12I illustrates a perspective view of an axially compressible seal positioned on the end of a male fitting.

FIG. 13 illustrates a side view of another embodiment of a connector of the present invention including an indicator to provide an indication to the user that the connector has been brought into connection with a cooperating fluid path connector with sufficient force to form an adequate connection therebetween.

FIG. 14 illustrates a side, cross-sectional view of an embodiment of a female fitting of a connector of the present invention including a deformable material that deforms upon contact with a cooperating male fitting.

FIG. 15 illustrates a side, cross-sectional view of an embodiment of a male fitting of a connector of the present invention including a radially deformable section on one end thereof that deforms upon contact with a cooperating female fitting.

FIG. 16 illustrates a side, cross-sectional view of an embodiment of a female fitting of a connector of the present invention including an annular elastomeric seal.

FIG. 17 illustrates a side, cross-sectional view of an embodiment of a male fitting of a connector of the present invention including an annular elastomeric seal.

FIG. 18 illustrates a side, cross-sectional view of an embodiment of a male fitting of a connector of the present invention including a radially outward projecting, circumferential seal or crush ring.

FIG. 19 illustrates a side, cross-sectional view of an embodiment of a female fitting of a connector of the present invention including a radially inward projecting sealing flange or crush ring.

FIG. 20 of the present invention includes a side, partially cross-sectional view of an embodiment of a connector of the present invention including a female fitting with an elastomeric sealing ring.

FIG. 21 illustrates a side, cross-sectional view of an embodiment of a connector of the present invention including a length of deformable tubing with a flange formed on an end thereof.

FIG. 22 illustrates a side, cross-sectional view of an embodiment of a connector of the present invention including a collet to form a sealed connection with a cooperating fluid path connector.

FIG. 23A illustrates a side, partially cross-sectional view of another embodiment of a connector of the present invention including a self-sealing seal in which the connector is in general alignment for connection to a cooperating connector of a syringe.

FIG. 23B illustrates a side, partially cross-sectional view of the connector of FIG. 23A in connection with the syringe.

FIG. 23C illustrates an enlarged, side, cross-sectional view of the seal of the female fitting of the connector of FIG. 23A.

### DETAILED DESCRIPTION OF THE INVENTION

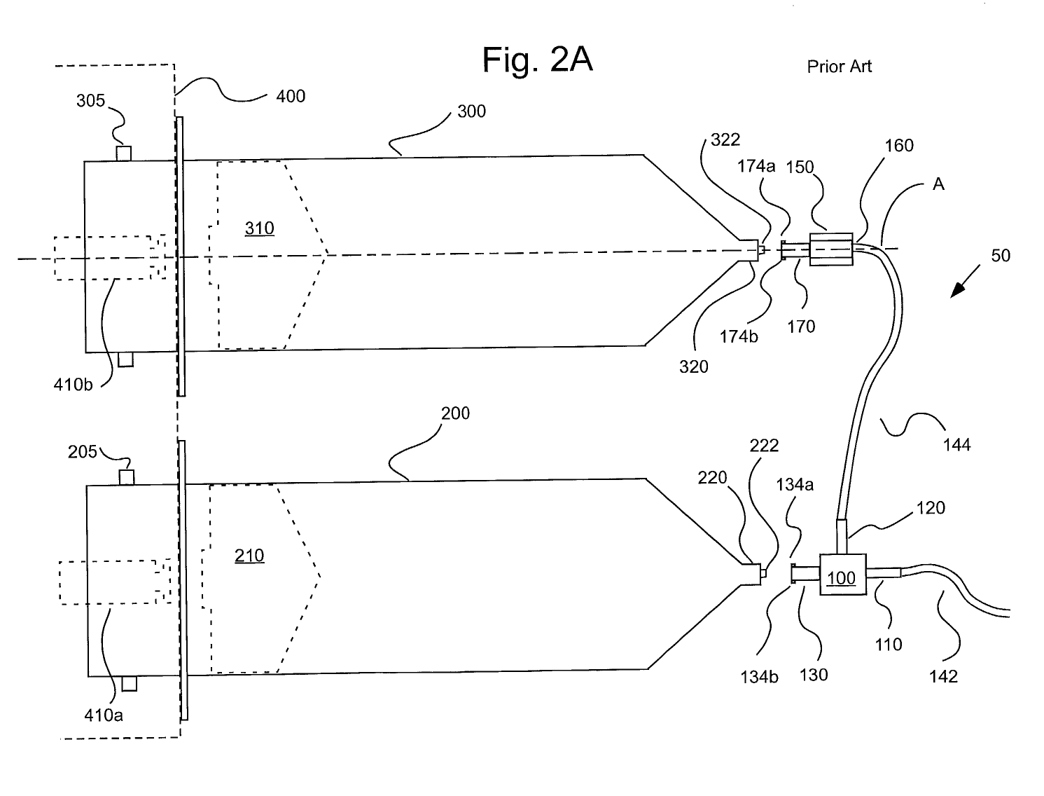

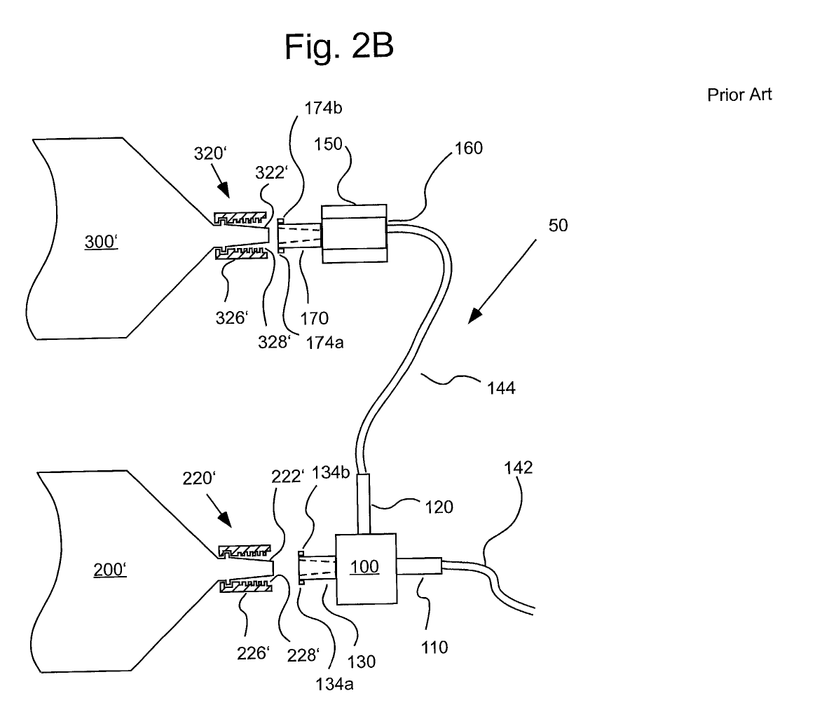

In one aspect, the present invention provides a syringe and fluid path connector for facilitating connection of, for example, tubing to the syringe. Before discussing this aspect of the present invention, two currently available syringe and fluid path connector systems are discussed in connection with FIGS. 2A and 2B to illustrate some of the problems that arise with current connectors. FIGS. 2A and 2B illustrate embodiments of fluid (for example, contrast media) delivery systems including two syringes.

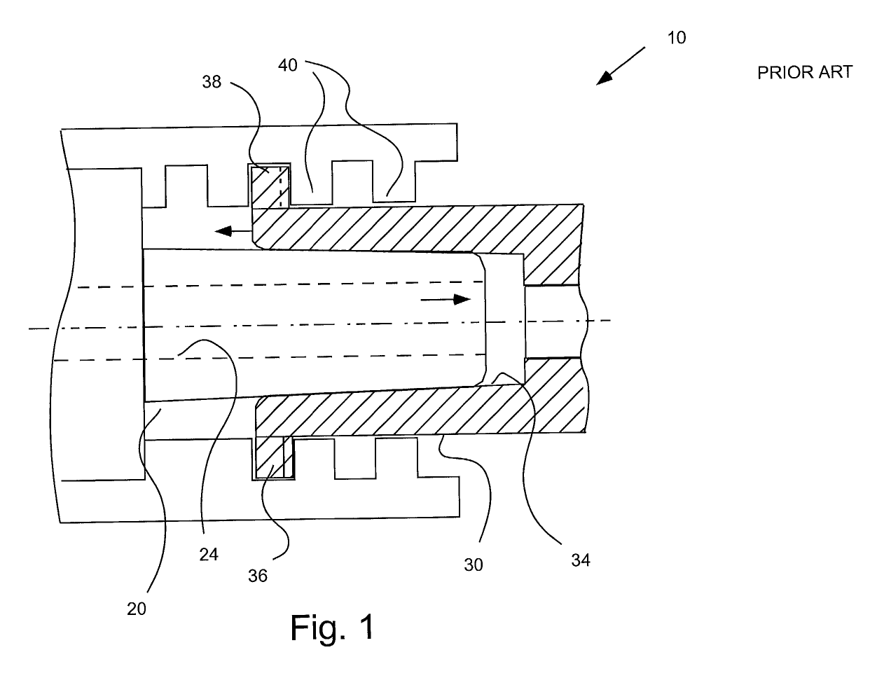

A fluid path 50 of fluid delivery system of FIG. 2A includes a T-connector 100 (which can, for example, include a valve mechanism) that includes a first port 110 and a second port 120 for connection of, for example, flexible tubing 142 thereto (via, for example, solvent welding). Connector 100 further includes a standard female luer fitting 130 having a tapered interior surface as known in the art. Female luer fitting or member 130 also includes flanges 134a and 134b on a rearward end thereof that cooperate with radially inwardly oriented threading (not shown in FIG. 2A, see FIG. 1) formed on a syringe tip 220 of a first syringe 200. Syringe tip 220 further includes a standard tapered male luer fitting 222 as described in FIG. 1. In general, luer fittings create a sealed connection via an “interference fit,” which refers generally to cooperating fittings in which the internal member or fitting is larger than the external member or fitting and has to be forced inside external member or fitting. In the case of a tapered fitting such as a luer fitting, the interference fit is created upon application of an axial compressive force to the cooperating/mating fittings.

As used herein as a convention in connection with the discussion of FIGS. 2A through 23C, the terms “axial” or “axially” refer generally to, for example, an axis A (or a similar axis) around which a second syringe 300 is preferably formed (although not necessarily symmetrically therearound) and to directions collinear with or parallel to such an axis. The terms “proximal” or “rearward” refer generally to an axial or a longitudinal direction toward the end of syringe 300 opposite a syringe tip 320 (from which pressurized fluid exits syringe 300). 2 5 The terms “distal” or “forward” refer generally to an axial or a longitudinal direction toward the syringe tip 320 of syringe 300. The term “radial” and related terms refers generally to a direction normal to an axis such as axis A.

First port 110 is in fluid connection with a patient (for example, via a length of flexible tubing 142 and other intervening fluid path elements). Second port 120 is connected, for example, via a length of flexible tubing 144, to a second connector 150 via a port 160 on a forward end of connector 150. Similar to connector 100, connector 150 further includes a standard female luer fitting 170 with a tapered interior surface. Female luer fitting or member 170 also includes flanges 174a and 174b on a rearward end thereof that cooperate with threading (not shown in FIG. 2A, see FIG. 1) formed on a syringe tip 320 of second syringe 300. As with syringe tip 220, syringe tip 320 further includes a standard tapered male luer fitting 322 as described in FIG. 1.

Each of syringe 200 and syringe 300 can, for example, be in removable operative connection with a powered injector 400 via, for example, attachment members 205 and 305 (for example, flanges) on a rearward portion of syringes 200 and 300, respectively. As known in the art, injector 400 includes at least one powered drive member that is adapted to cooperate with a plunger slidably disposed within a connected syringe to impart reciprocal sliding motion to the plunger to either draw fluid into the syringe (retracting motion) or to expel fluid from the syringe (advancing motion). In the embodiment of FIG. 2A, injector 400 includes two drive member 410a and 410b that cooperate with plungers 210 and 310, respectively. An example of a powered injector and syringe suitable for use in the present invention is the Spectris® Injector available from Medrad, Inc. of Indianola, Pa.

During connection of fluid path 50 to syringes 200 and 300, connector 100 can, for example, be first threaded onto syringe tip 220 via flanges 134a and 134b of female luer fitting 130 to form a connection suitable to withstand the relatively high pressure generated during an injection procedure. Subsequently, second connector 150 can be similarly threaded onto syringe tip 320 via flanges 174a and 174b of female luer fitting 170. Difficulties can arise in forming a connection between second connector 150 and syringe tip 320 because such connection requires rotation of connector 150 to which tubing 144 is attached.

FIG. 2B illustrates another embodiment of a currently available fluid delivery system for use, for example, in angiography, MRI or CT. As in the embodiment of FIG. 2A, fluid path 50 is connectable to syringes 200′ and 300′. Unlike syringe 200 and 300, however, in which the threading of the syringe tips thereof is stationary. Syringe tips 220′ and 320′ include threaded swivel nuts 226′ and 326′ that are rotatable relative to male luer fittings 222′ and 322′, respectively. In this embodiment, first connector 100 can, for example, be first threaded onto syringe tip 220′ via female luer fitting 130 by appropriately positioning flanges 134a and 134b to come into contact with threading 228′ of swivel nut 226′. Swivel nut 226′ is the rotated about its axis to form a connection between first connector 100 and syringe 200′. Subsequently, second connector 150 is connected to syringe 300′ by cooperation of threading 328′ of swivel nut 326′ with flanges 174a and 174b. Because the connection between syringe 300′ and second connector 150 is made by rotation of swivel nut 326′ relative to second connector 150 rather than rotation of second connector 150, attachment is made easier compared to the embodiment of FIG. 2A. In that regard, tubing144 connected to second connecter 150 need not be rotated/twisted, thereby avoiding the resistance to connection that can arise from such rotation/twisting.

Although the embodiment of FIG. 2B provides some improvement in operation as compared to the embodiment of FIG. 2A, a number of problems persist. For example, two hands are typically required to connect the male luer fitting of syringes 200′ and 300′ with the female luer fittings of connectors 100 and 150. Moreover, placement of swivel nuts 226′ and 326′ on syringes 200′ and 300′, respectively, substantially increases production difficulty and production cost of syringes 200′ and 300′. Additionally, operators often damage or break luer fittings of the type illustrated in FIGS. 2A and 2B as well as other types of luer fittings in attempting to form a secure connection.

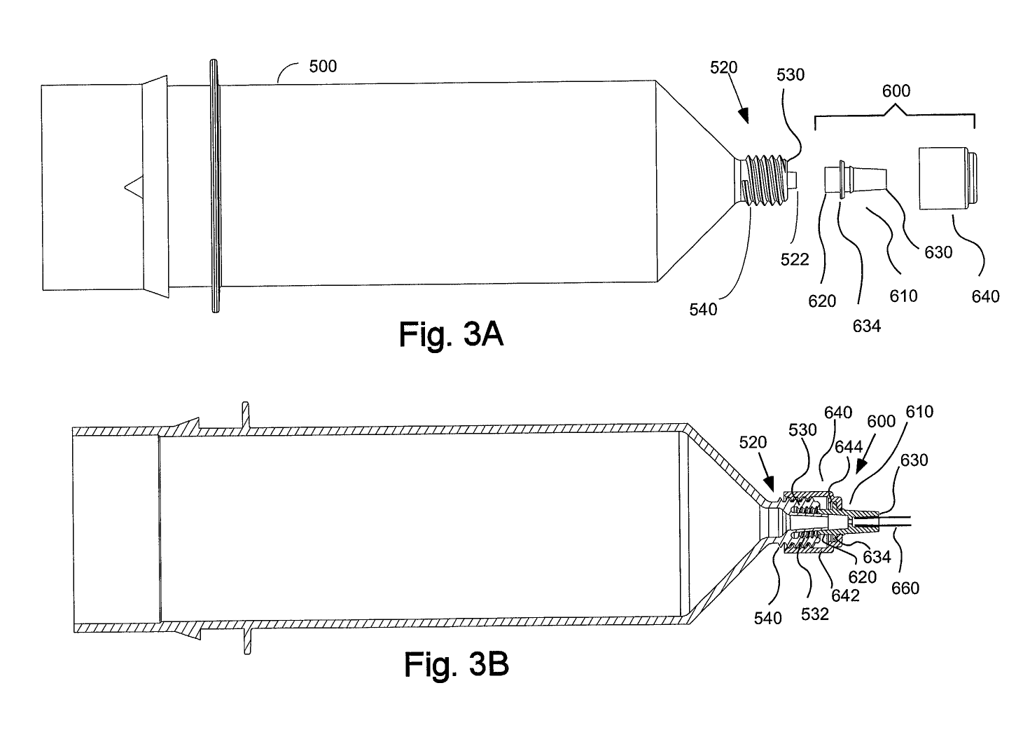

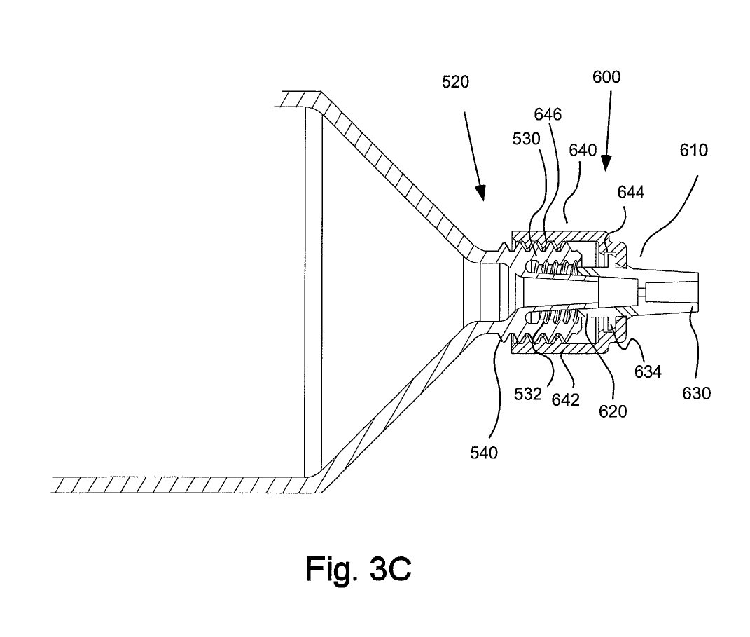

FIG. 3A through 3C illustrate one embodiment of a syringe 500 and a cooperating fluid path connector element 600 that overcome many of the above-identified problems with currently available standard luer connections. Syringe 500 includes a syringe tip 520 that includes a tapered male (for example, luer-type) fitting 522. Syringe tip 520 includes a generally cylindrical member 530 surrounding male fitting 522. Surrounding member 530 preferably includes radially inwardly directed threading 532 (see FIGS. 3B and 3C) to cooperate with the flange(s) of a tapered female fitting (for example, a standard female luer fittings such as female luer fittings 130 or 170 described above). Surrounding member 530 also preferably includes at least one “outer” attachment member or abutment member 540 such as a radially outward extending flange. In the embodiment of FIGS. 3A through 3D, attachment member 540 includes a helical flange that forms radially outward extending threading.

A mating or cooperating fluid path connection element 600 preferably includes a conduit member 610 having a tapered female fitting 620 on a rearward end thereof which is in fluid connection with a port 630 on a forward end of conduit member 610. Port 630 can, for example, be connected to tubing 660 via, for example, solvent welding. Female fitting 620 and port 630 can, for example, be fabricated from an integral piece of polymeric material. Connection element 600 also includes an attachment member 640 that is adapted to cooperate with attachment member 540 of syringe tip 520 to place connector 600 and syringe 500 in fluid connection. In the embodiment of FIGS. 3A through 3C, attachment member 640 includes a rotating element or swivel nut 642 that is rotatably attachable to conduit member 610 via cooperation of a flange 634 on conduit member 610 and a cooperating groove 644 formed in rotating element 642. Rotating element 642 includes a generally cylindrical rearward portion that includes radially inward projecting threading 646.

During connection of connector 600 to syringe 500, female fitting 620 is brought into general alignment with male fitting 522. Rotating element 642 is rotated about its axis relative to syringe tip 520. Threading 646 cooperates with threaded attachment member 540 to bring connector 600 and syringe 500 into sealing fluid connection.

Unlike currently available luer connectors as, for example, described above in connection with FIGS. 1, 2A and 2B, syringe 500 and fluid path connector 600 can be easily connected using only one hand. In that regard, the user can use one hand to both bring female fitting 620 into general alignment with mating male fitting 522 and to rotate element 642 relative to syringe tip 520. Moreover, rotating element 642 on connector 600 prevents rotation/twisting of attached tubing 660. Furthermore, syringe 600 is less expensive to manufacture than syringes 200′ and 300′ and can remain usable with currently available, standard female luer fittings (as described, for example, in FIGS. 1 through 2B) via threading 532.

The present invention also provides other connectors suitable for use with the syringes of the present invention as well as with other fluid path elements or fluid pumping systems. In general, the connectors of the present invention are suitable for use in low pressure and high-pressure procedures. In that regard, pressures in injection procedures such as contrast enhance CT scans can exceed approximately 300 psi, while pressures in angiographic procedures can exceed approximately 1200 psi.

In several embodiments, the connectors of the present invention provide connectors that include mating fittings that are brought together to form an interference fit. The fitting can, for example, be male and female tapered fittings such as luer fittings.

However, in the case of tapered fittings, the taper need not be 6% as specified for standard luer fittings. Moreover, the taper on the male fitting and the taper on the female fitting can be different (for example, 6% and 12%). In several such embodiments, a compressive axial force is maintained between the cooperating fittings after mating of the cooperating fittings.

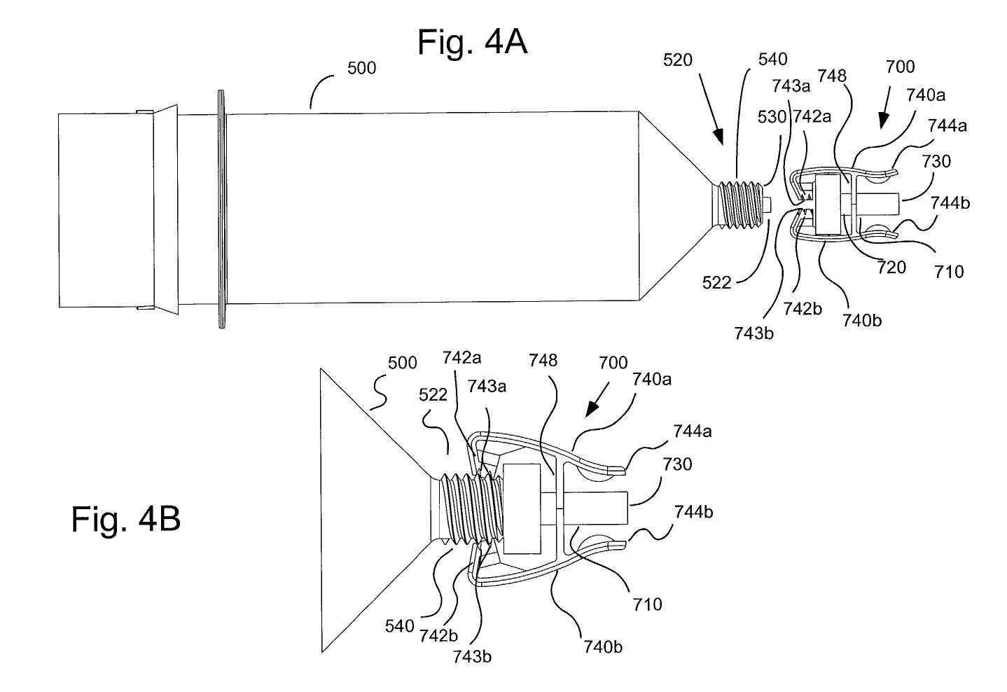

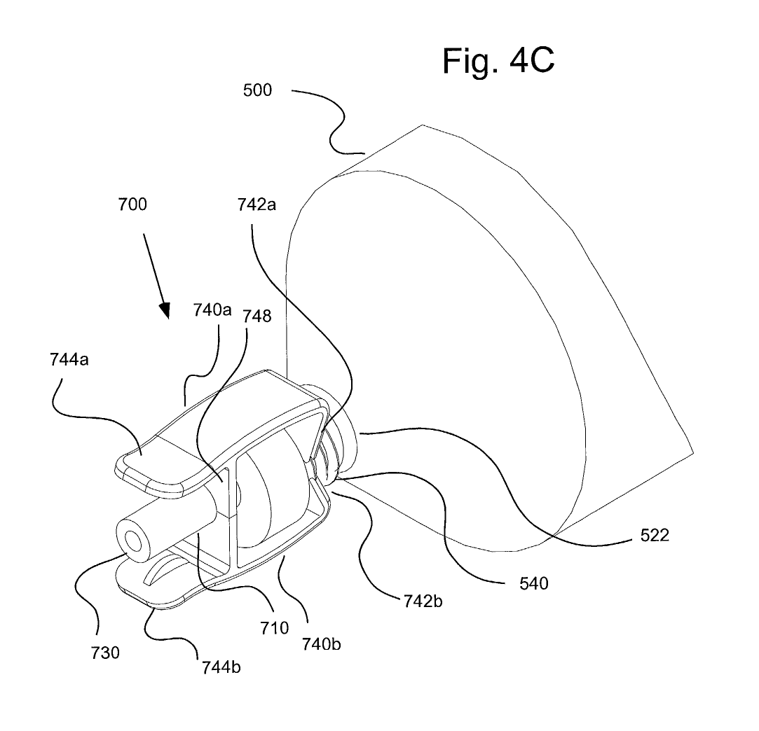

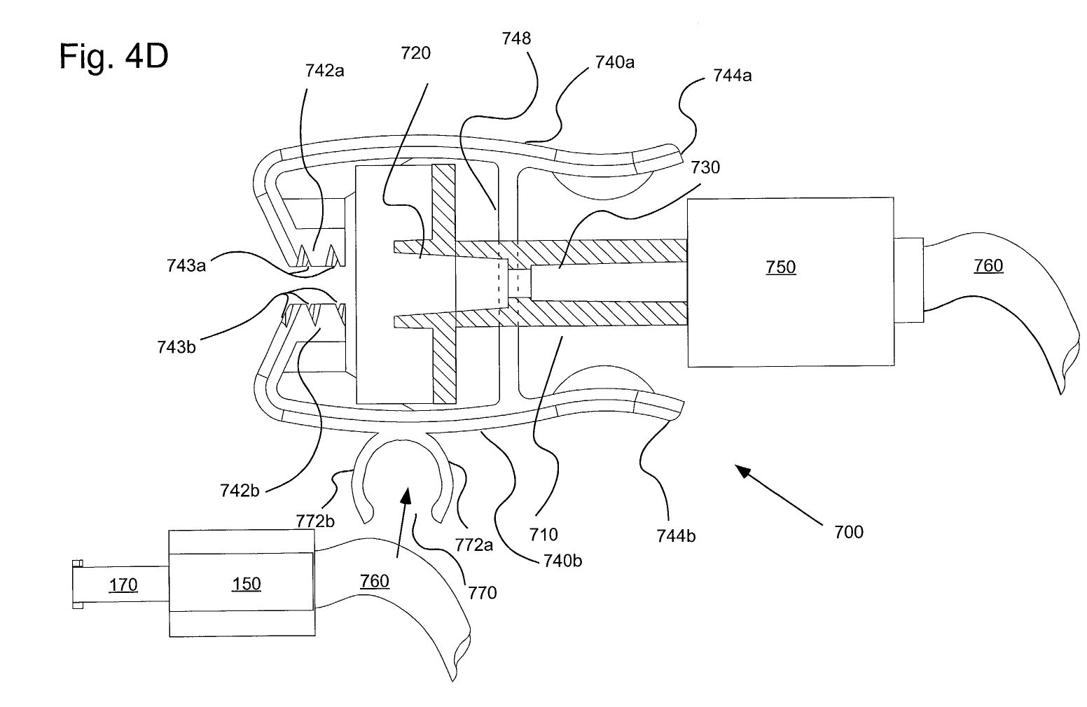

For example, FIGS. 4A through 4D illustrate a connector 700 that is attachable to, for example, syringe 500. Connector 700 includes a conduit member having a tapered female fitting 720 (for example, a female luer fitting) on a rearward end thereof, which is in fluid connection with a port 730 on a forward end thereof. Fluid path connector 700 also preferably includes a cooperating attachment member to form a connection with attachment member 540 of syringe 500. In the embodiment of FIGS. 4A through 4D, the cooperating attachment member includes a pair of rearward extending, resilient, flexible, gripping arms 740a and 740b connected via a bridging member 748.

The rearward ends of gripping arms 740a and 740b include gripping elements or radially inward extending abutment flanges 742a and 742b that, in the embodiment of FIGS. 4A through 4C are adapted to mate with threaded attachment member 540. In that regard, gripping elements 742a and 742b can include grooves 743a and 743b (see, for example, FIGS. 4B and 4D), respectively, that are adapted (for example, appropriately oriented and dimensioned) to cooperate or mate with the threads of threaded attachment member 540 when connector 700 is attached to syringe tip 520.

During connection of connector 700 to syringe 500, forward ends 744a and 744b of gripping arms 740a and 740b , respectively, can be forced radially inward by the user, causing the rearward ends of gripping arms 740a and 740b to move radially outward to pass over/around syringe tip 520. Preferably, the rearward surfaces of gripping arms 740a and 740b are angled or sloped forward to, for example, facilitate connector 700 passing rearward over syringe tip 520 (as discussed in greater detail below in connection with connector 800). Once contact/connection has been made between, tapered female fitting 720 and tapered male fitting 522 (for example, a male luer fitting), the user can release his or her compression of forward ends 744a and 744b of gripping arms 740a and 740b, thereby causing gripping elements 742a and 742b to flex inward to come into contact with threaded attachment member 540.

As illustrated, for example, in FIG. 4B, grooves 743a and 743b form a mating attachment with the threads of attachment member 540 so that when the user rotates connector 700 about its axis relative to syringe 500, for example, approximately 90° or approximately ¼ turn in a clockwise direction, the mating connection between female fitting 720 and male fitting 522 is brought under additional axial compressive force to provide and maintain a sealing connection. Preferably, a plurality of grooves 743a and a plurality of grooves 743b form a mating connection with the threads of attachment member 540. Moreover, as clear to one skilled in the art, the angle of orientation of a radially inward surface of gripping elements 742a and 742b in which grooves 743a and 743b are formed can be adjusted such that mating contact of grooves 743a and 743b with threaded attachment member is maximized when connector 700 is attached to syringe tip 520.

Unlike currently available luer connectors, the user cannot overtighten the connection (which often leads to breakage in currently available luer connectors) between connector 700 and, for example, syringe 500. In that regard, if the user continues to tighten the attachment between connector 700 and syringe 500 by rotating connector 700, eventually a radial component of the resultant force on connector 700 will cause gripping elements 742a and 742b to open to a radial outward position and move or slip forward over a portion of threaded attachment member 540 until force on connecting member 700 is reduced and gripping elements 742a and 742b can once again flex radially inward to grip attachment member 540. This “slippage” assists in preventing over tightening.

As illustrated in FIG. 4D, connector 700 and other connectors of the present invention can also include an additional fluid path element such as a multiport connector or a valve mechanism 750 (for example, a check valve, a ball valve, a stopcock valve, other type of valve, transducer or other fluid path element) in fluid connection with port 730. Valve 750 can include one or multiple ports for connection to other fluid path elements. In FIG. 4D, valve 750 includes one port to which flexible tubing 760 is attached. Connector 700 and other connectors of the present invention can also include a retainer 770 for preferably removably attaching, for example, tubing 760 or another fluid path element in fluid connection with port 730 (either directly or via, for example, valve 750). In FIG. 4D, retainer 770 includes a first flexible, resilient arm 772a and a second flexible, resilient arm 772b into which tubing 760 can be snap fit so that resilient arms 772a and 772b surround the circumference of tubing 760. The effective diameter of retainer 770 is preferably less than the diameter of tubing 760 such that resilient arms 772a and 772b apply radial inward pressure upon the outer wall tubing 760. Retainer 770 can be used, for example, to assist in positioning tubing or in preventing contamination of, for example, connector 150 (including, for example, a female luer fitting 710 as described in connection with FIGS. 2A and 2B) that, without retainer 770, could trail behind connector 700 in an uncontrolled manner. Valve 750 and retainer 770 can, for example, be formed integrally with connector 700.

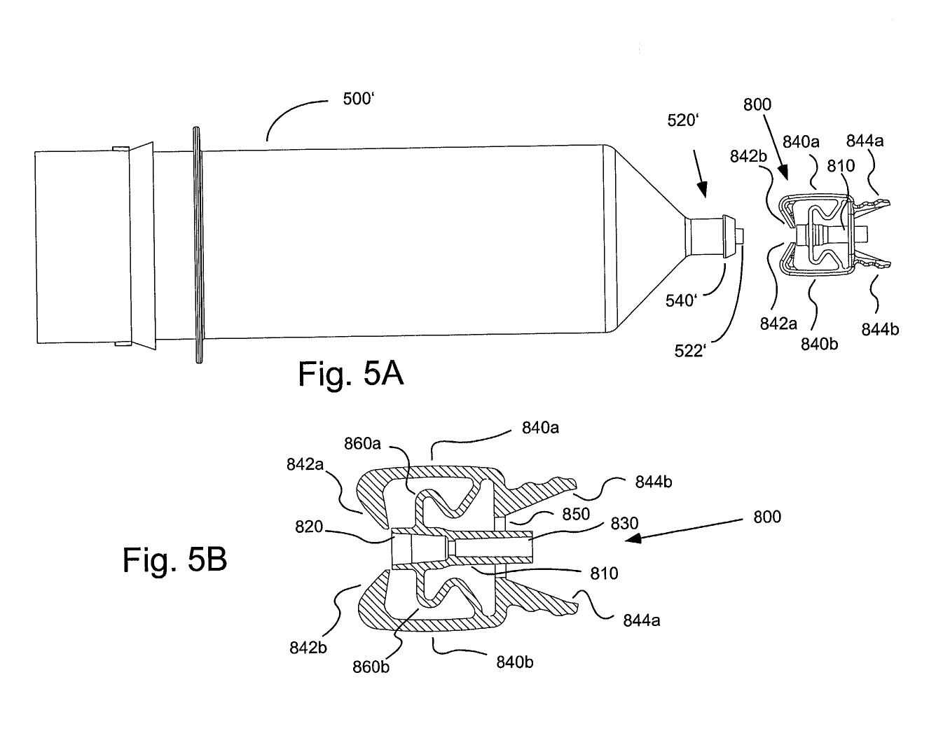

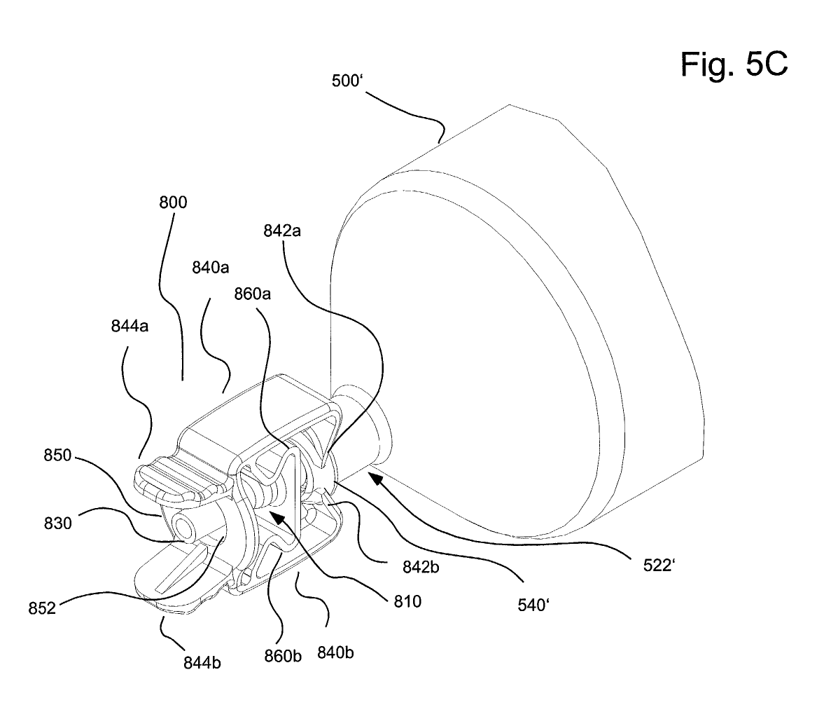

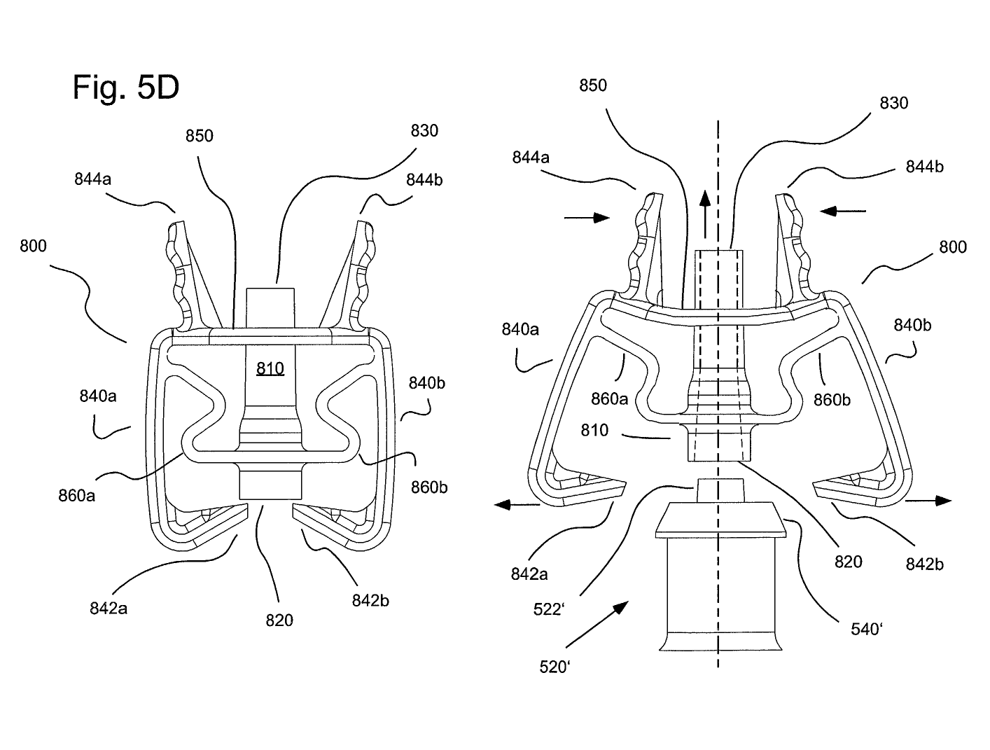

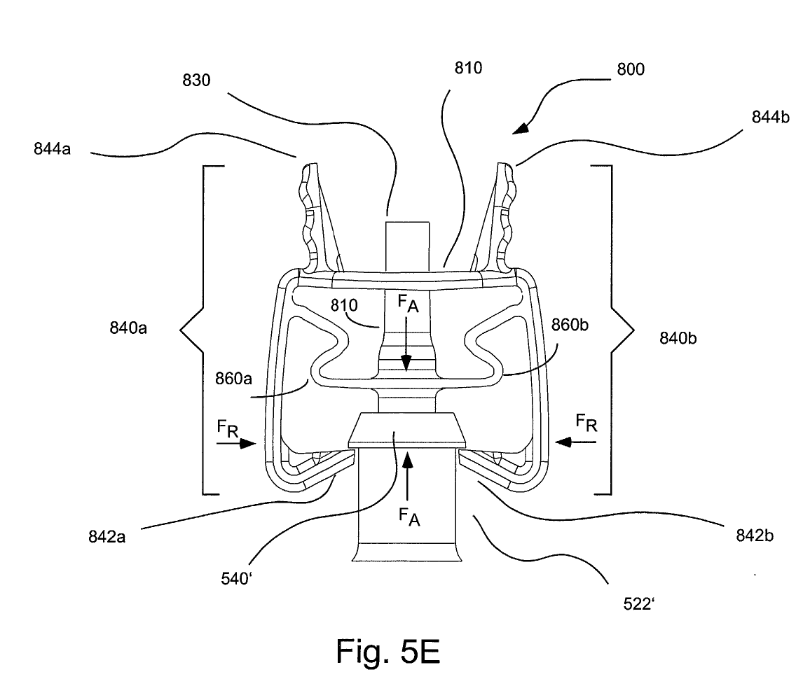

FIGS. 5A through 5E illustrate another embodiment of a connector 800 of the present invention. In the case of a connector 700 described above, the forces maintaining sealed fluid connection between connector 700 and syringe 500 are the generally radially inward force exerted by gripping elements 742a and 742b upon threaded attachment member 540 and the generally axially compressive force caused by the interaction of grooves 743a and 743b with the threads of threaded attachment member 540 as described above. To ensure an acceptable sealed connection, connector 700 is preferably manufactured to tolerances that are fairly tight. Connector 800 enables loosening of such tolerances while also providing an axial compressive force tending to maintain connector 800 in substantially sealed fluid connection with syringe 500′.

Syringe 500′ includes a syringe tip 520′ which includes a tapered male fitting 522′ (for example, a male luer fitting) as described above for syringe 500. Syringe tip 520′ also includes an attachment member 540′ which includes a radially outward extending flange in the embodiment of syringe 500′. Connector 800 preferably includes a conduit member 810 that includes a tapered female fitting 820 (for example, a female luer fitting) on a rearward end thereof, which is in fluid connection with a port 830 on a forward end thereof. Fluid path connector 800 also preferably includes cooperating attachment members to form a connection with attachment member 540′ of syringe 500′ (or with, for example, attachment member 540 of syringe 500).

Similar to connector 700, the cooperating attachment member of connector 800 includes a pair of rearward extending, resilient gripping arms 840a and 840b.

The rearward ends of gripping arms 840a and 840b include cooperating attachment members or gripping elements 842a and 842b that are adapted to engage, for example, flanged attachment member 540′. In the embodiment of FIGS. 5A through 5E, gripping elements 842a and 842b are radially inward extending shoulders or flanges formed on the rearward end of connector 800.

As illustrated best in FIGS. 5C through 5E, during connection of connector 500 to, for example, syringe 500 or 500′, the forward ends 844a and 844b of gripping arms 840a and 840b , respectively, can be pressed radially inward by the user, causing the rearward ends of gripping arms 840a and 840b to move radially outward to pass over/around syringe tip 520′. In the embodiment of FIGS. 5A through 5E, gripping arms 840a and 840b are connected via a bridging member 850 which bends or bows (as best illustrated in FIG. 5D) when forward ends 844a and 844b of connector 800 are forced radially inward and/or when gripping elements 842a and 842b are forced radially outward. Once connection has been made between female fitting 820 and male fitting 522′, the user releases his or her compression of forward ends 844a and 844b of gripping arms 840a and 840b so that gripping elements 842a and 842b flex inward to engage attachment member 540′.

Preferably, the rearward surfaces of gripping elements 842a and 842b are angled or sloped in a forward direction (traveling from a radial outer point to a radial inner point) to facilitate connector 800 passing rearward over syringe tip 520′. Likewise, flange 540′ can be angled or sloped in a rearward direction to facilitate connection. In this manner, it is not generally necessary that forward ends 844a and 844b of connector 800 be squeezed or forced radially inward by the user during connection. The user can simply impart rearward motion to connector 800. As the sloped rearward surfaces of gripping elements 842a and 842b contact the sloped surface of flange 540 (as illustrated in FIG. 5C), gripping elements 842a and 842b are forced radially outward to allow flange 540′ to pass therebetween. Once flange 540′ passes between gripping elements 842a and 842b, gripping arms 840a and 840b preferably snap back toward an unstressed state so that gripping elements 842a and 842b move radially inward to form a mating abutment connection with flange 540′. The snapping back of gripping arms 840a and 840b can provide audible and/or tactile feedback to the user to indicate a secure connection. The bending or flexing moment(s) about the center of bridging member 850 and/or about gripping arms 840a and 840b provide a radially inward force upon each of gripping elements 842a and 842b to maintain gripping elements 842a and 842b in connection with flange 540.

In addition to the radially inward force upon gripping elements 842a and 842b, connector 800 also can provide an axially compressive force between connector 800 and syringe tip 520′ to assist in maintaining a fluid connection therebetween. In the embodiment of FIGS. 5A through 5E, connector 800 includes a biasing element to bias male fitting 522′ and female fitting 820 in a connected state. The biasing element of FIGS 5A through 5E includes a first curved, resilient spring arm 860a connecting gripping arm 840a to conduit member 810 and a second curved, resilient spring arm 860b connecting gripping arm 840b to conduit member 810.

When gripping elements 842a and 842b move radially outward (whether through radially inward compression of forward ends 844a and 844b or contact of gripping elements 842a and 842b with flange 540 as described above) spring arms 860a and 860b come under tension and lengthen or become less curved as illustrated in the right-hand portion of FIG. 5D. Conduit member 810 attached to spring arms 860a and 860b is free to move in an axial direction relative to bridging member 850. In that regard, bridging member 850 preferably includes a passage 852 (see FIG. 5C) formed therein to allow axial motion of conduit member 810 relative to bridge member 850. The motion of gripping arms 840a and 840b and conduit member 810 upon, for example, compression of forward ends 844a and 844b and rearward movement of connector 800 to contact syringe tip 520′ is represented by arrows in the right-hand portion of FIG. 5D. Spring arms 860a and 860b also add rigidity to gripping arms 840a and 840b and provide a radially inward force component to assist in maintaining gripping arms 840a and 840b in operative connection with flange 540′.

When gripping elements 842a and 842b move radially inward to form a connection with flange 540′ as illustrated, for example, in FIG. 5E, and gripping arms 840a and 840b move toward their non-stressed, resting state. Because gripping arms 840a and 840b are prevented from returning completely to the non-stressed, resting state (illustrated, for example, in the left-hand portion of FIG. 5D) by contact of gripping elements 842a and 842b with syringe tip 520′, the resultant bending moment about bridging member 850 and/or about gripping arms 840a and 840b imparts a radially inward force component F_{R} exerted upon gripping elements 842a and 842b as described above. Likewise, spring arms 860a and 860b are prevented from returning to a non-stressed, resting state by contact between male fitting 522′ and female fitting 820 and by abutment of a forward surface of gripping elements 842a and 842b with a rearward shoulder or surface of flange 540′. Spring arms 860a and 860b cause an axially compressive force component F_{A} to be exerted between female luer fitting 820 of connector 800 and male luer fitting 522′ of syringe tip 520′. Axial force component F_{A} assists in maintaining a sealed fluid connection between connector 800 and syringe 500′ while assisting in loosening tolerances required in the manufacture of connector 800 as compared to connector 700 in which generally only radial force is used to maintain a fluid connection.

The biasing members or spring arms of the present invention can take a variety of forms other than as illustrated in FIGS. 5A through 5E. For example, resilient bridging member 748 of connector 700 can act as a biasing member or spring arm if positioned such that is placed in a stressed state when connector 700 is engaged to syringe 500′.

When connector 800 is connected to syringe tip 520′, connector 800 can be rotated about its axis relative to syringe tip 520 to any orientation without comprising the sealed engagement of male luer fitting 522′ and female luer fitting 720.

Connectors 700 and connector 800 (as well as other connectors of the present invention described below) can, for example, be fabricated from a resilient, polymeric material such as a polycarbonate, polyethylene teraphthalate, ABS, or polyurethane via, for example, an injection molding process. Preferably, connector 700 and connector 800 are formed from an integral piece of such polymeric material via injection molding. Molding the connectors of the present invention (or components thereof) from an integral piece of polymeric material in a single molding process reduces production cost, time and complexity as compared to many currently available connectors. Suitable polymeric materials, for example, preferably exhibit a flexural modulus in the range of approximately 200,000 to approximately 400,000 psi. Such polymeric materials also preferably exhibit a tensile strength at yield of approximately 6000 to approximately 10,000 psi. Furthermore, such polymeric materials are also preferably suitable for solvent bonding with flexible tubing used in medical procedures (typically, polyvinyl chloride or PVC). The connectors of the present invention can also be molded or over-molded with, for example, flexible tubing in place to bond the flexible tubing thereto.

Using such materials in the connectors 700 and 800 and other connectors of the present invention provides excellent durability and enables use of the connectors at relatively high pressures experienced, for example, in injection procedures with powered injectors. Moreover, as clear to one skilled in the art, minor design changes such as material thickness and flexural modulus can be adjusted to increase the pressure at which the connectors of the present invention can be used.

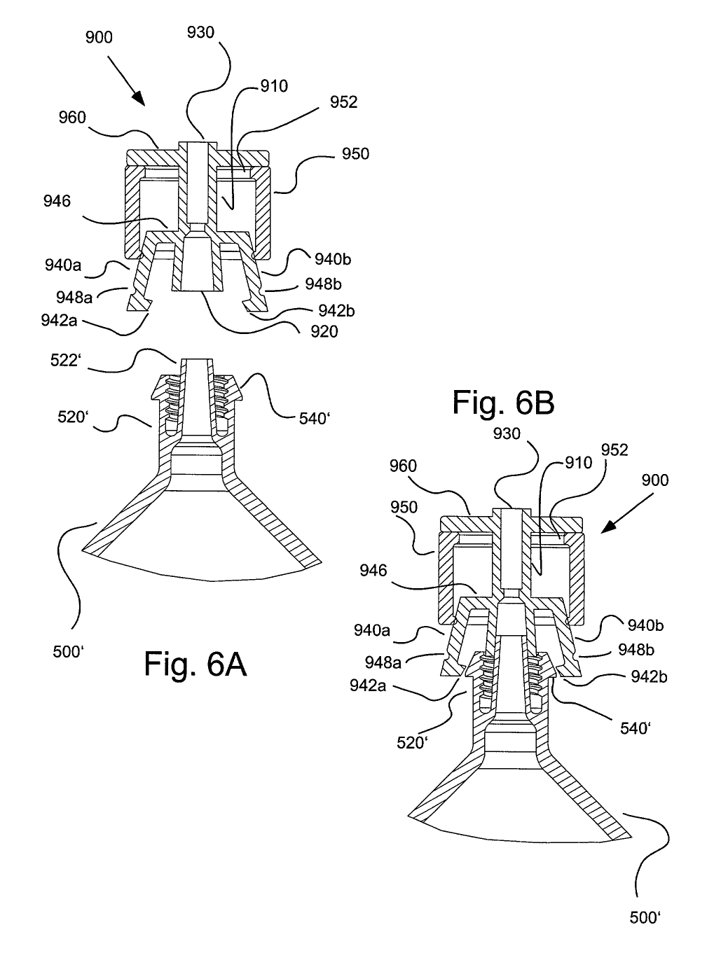

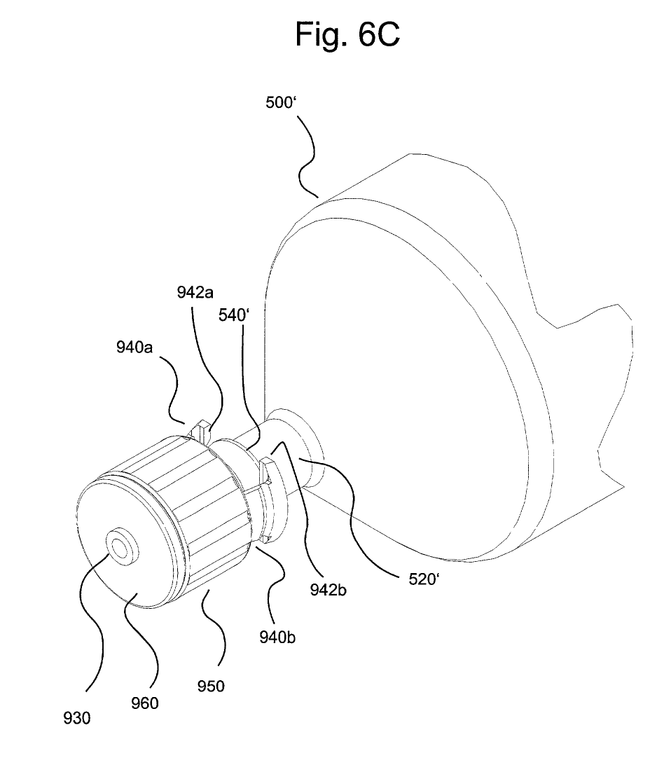

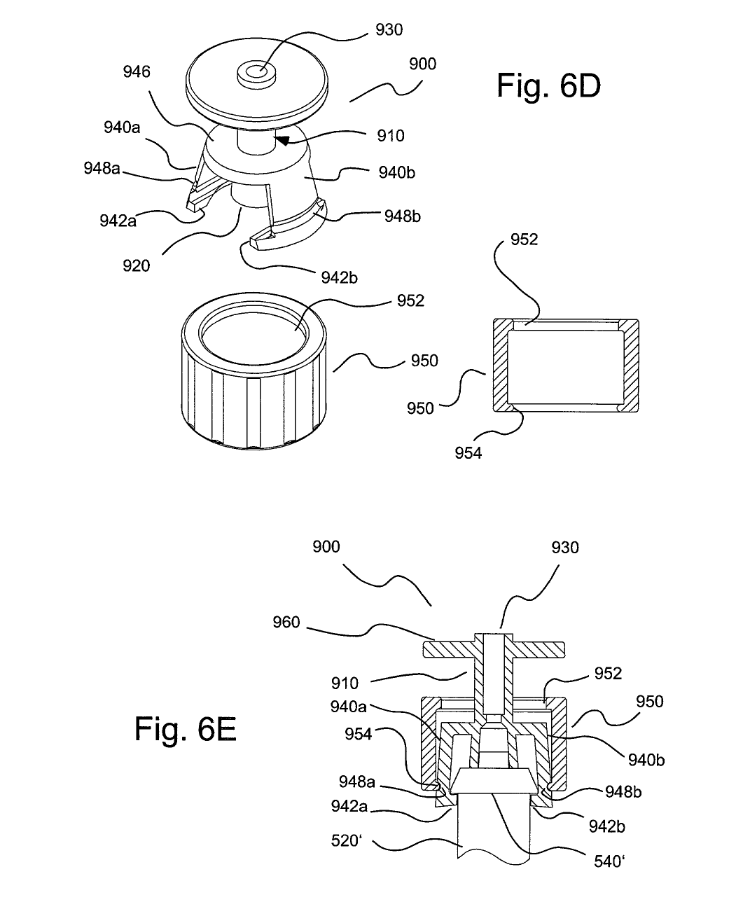

FIGS. 6A through 6E illustrate another embodiment of a connector 900 of the present invention. Like connectors 700 and 800, connector 900 include a conduit member 910 that can include a tapered female fitting 920 (for example, a female luer fitting) on a rearward end thereof and at least one port 930 on a forward end thereof. Also like connectors 700 and 800, connector 900 also includes at least two resilient, flexing, arms 940a and 940b extending rearward. Gripping elements 942a and 942b are positioned on a rearward end of gripping arms 940a and 940b , respectively. Gripping element 942a and 942b cooperate with, for example, attachment flange 540′ on syringe 500′ (or, for example, with threaded attachment member 540 of syringe 500) as discussed above in connection with gripping elements 842a and 842b of connector 800. Gripping elements 940a and 940b are preferably attached to conduit member 910 via attachment member 946.

Connector 900 also includes a generally cylindrical locking member, collar or collet 950 that is slidably disposed upon connector 900 via a passage 952 (see, for example, FIG. 6D) formed in a forward surface of locking member 950. Locking member 950 is retained upon connector 900 by an abutment with forward abutment flange 960 attached to a forward portion of conduit member 910 and by abutment with gripping arms 940a and 940b to the rear.

During connection of connector 900 to syringe 500′, female fitting 920 of connector 900 is first aligned with a mating or cooperating tapered male fitting 522′ (for example, a male luer fitting) of syringe tip 520′ with locking member 950 in a forward position as illustrated in FIG. 6A. Connector 900 is then moved rearward relative to syringe 500′ to bring female fitting 920 and male fitting 522′ into contact (see, FIGS. 6B and 6C). Gripping elements 942a and 942b pass to the rear of attachment flange 540′ once female fitting 920 and male fitting 522′ are brought into full contact. With the forward surfaces of gripping elements 942a and 942b positioned to the rear of the rearward surface of attachment flange 540′, locking element 950 is moved to a rearward position as illustrated in FIG. 6E so that gripping arms 940a and 940b are forced radially inward and gripping elements 942a and 942b are brought into engagement with attachment flange 540′. A rearward portion of locking member 950 can include a radially inwardly projecting flange 954 that cooperates with grooves 948a and 948b formed in gripping arms 940a and 940b, respectively, to assist in retaining locking member 950 in the rearward, engagement position. The cooperation of flange 954 and grooves 948a and 948b can also provide audible and/or tactile feedback to the user to indicate a secure connection.

Gripping elements 942a and 942b in cooperation with flange 540′ can provide an axially compressive force as described above. For example, gripping elements 942a and 942b can be angled or sloped forward such that a bending moment is created about gripping elements 942a and 942b when in contact with the rear surface of flange 540′.

To release connector 900 from syringe 500′, locking member 950 is slid to its forward or disengagement position (as illustrated in FIGS. 6A and 6B) to allow gripping arms 940a and 940b to swing or flex radially outward and disengage gripping elements 942a and 942b from attachment flange 540′. Connector 900 is preferably fabricated from a resilient polymeric material as described above.

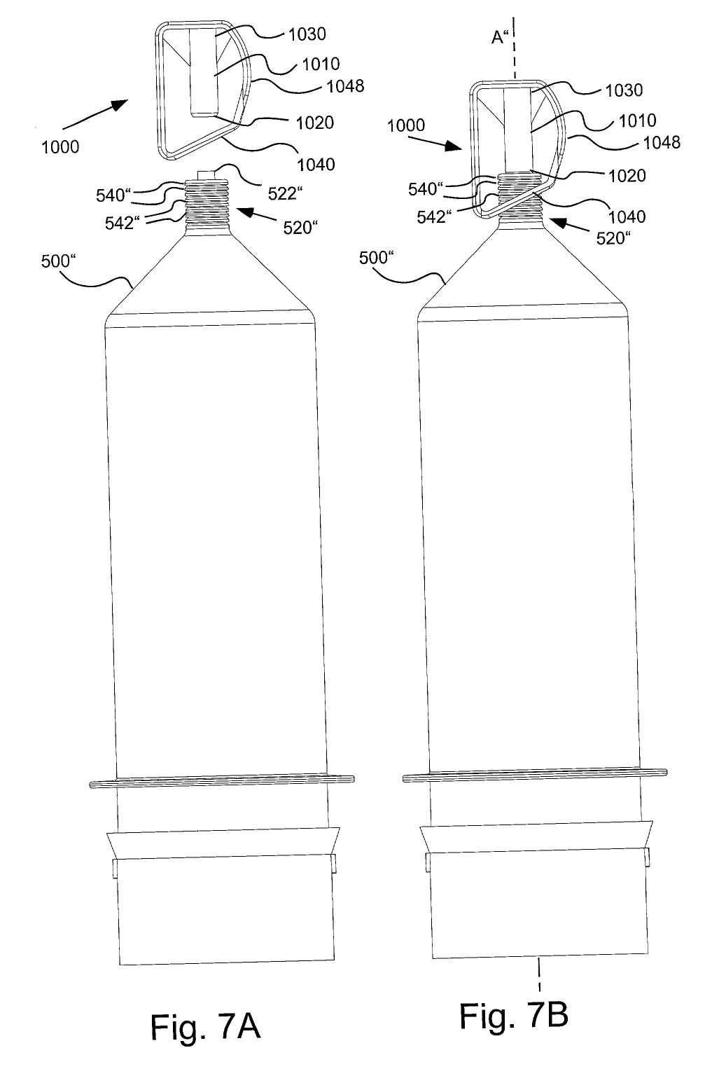

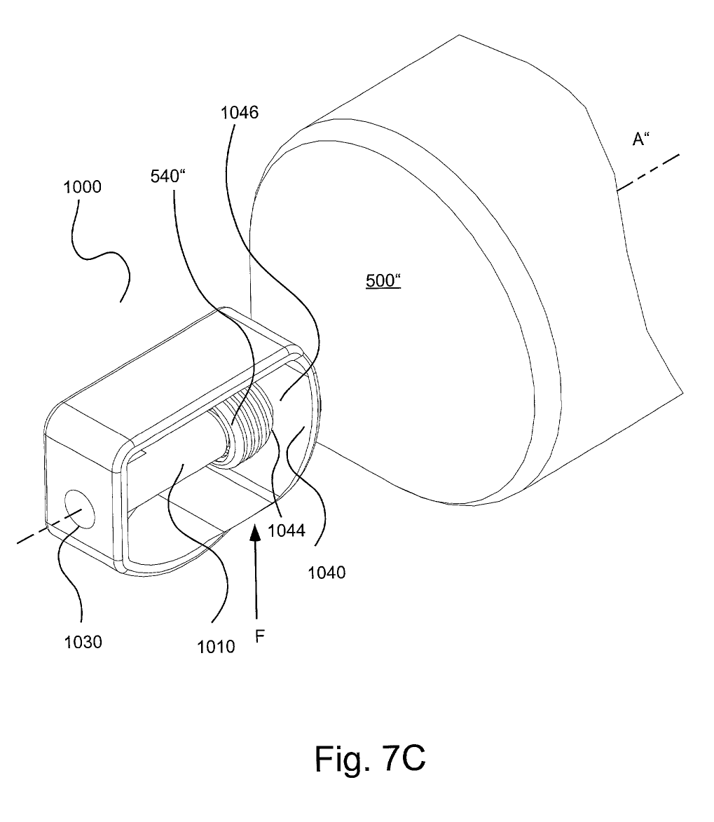

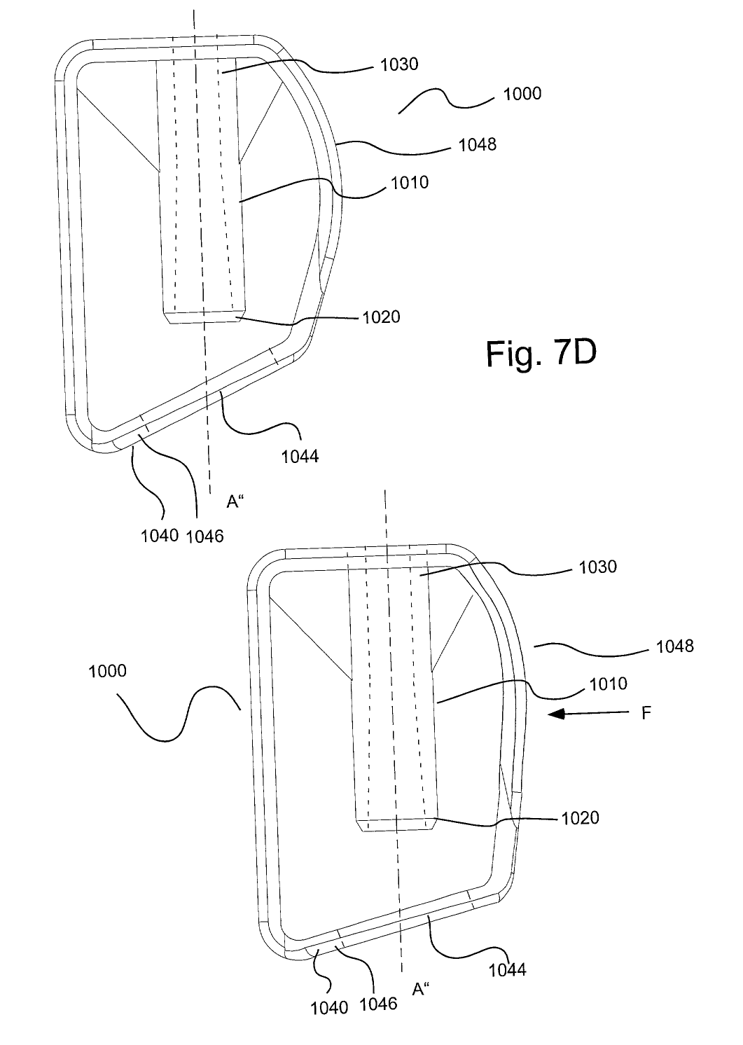

FIGS. 7A through 7D illustrate a further embodiment of a connector 1000 of the present invention. Connector 1000 includes a conduit member 1010 including a tapered female fitting 1020 (for example, a female luer fitting) and a port 1030 in fluid connection with female fitting 1020 as described above. Connector 1000 also preferably includes a gripping element 1040 to cooperate with, for example, attachment flanges 540″ (and intermediate grooves 542″) on syringe tip 520″ of syringe 500″ (or, for example, with attachment flange 540′ of syringe 500′ or with threaded attachment member 540 of syringe 500) to attach connector 1000 to syringe tip 520″.

In the embodiment of FIGS. 7A through 7D, gripping element or cooperating attachment member 1040 includes a passage 1044 formed therein. In a first, engaged position illustrated, for example, in FIGS. 7A and 7B and the upper portion of FIG. 7D, member 1040 and passage 1044 are oriented at an angle to axis A″ of syringe 500″ so that the effective area of passage 1044 (as, for example, projected on a plan perpendicular to axis A″) is reduced and at least a portion of the perimeter of passage 1044 (defining the interior radius/surface of a radially inward projecting flange 1046) engages attachment flanges 540″ and intermediate grooves 542″ such that connector 1000 resists removal from syringe tip 520″ (see FIG. 7B). During connection and removal of connector 1000, the orientation of member 1040 (and, thereby, passage 1044 and flange 1046) is changed in a manner to increase the effective area/diameter of passage 1044 to enable passage 1044 (and generally circumferential flange 1046 defined thereby) over attachment flanges 540″.

In connector 1000, the orientation of passage 1044 and flange 1046 is controlled by the state of at least one flexing element such a curved flexing element 1048. As illustrated in FIGS. 7C and 7D, force F is applied to flexing element 1048 (as represented by the arrows of FIGS. 7C and 7D) to reduce the curvature thereof and thereby to change the orientation of passage 1040 to be closer to perpendicular to axis A″, thereby increasing the effective area of passage 1044 with respect to axis A″ and allowing flange 1046 to pass over attachment member(s) 540″. Connector 1000 is preferably fabricated from a resilient polymeric material as described above, and preferably from an integral piece thereof.

More than one transverse member 1040, passage 1044 and flange 1046 can be provided to improve the connection between connector 1000 and a mating or cooperating fitting assembly (for example, syringe 500″). Furthermore, connector 1000 can connect to a fitting assembly that does not include one or more radially extending flanges such as attachment members 540″. For example, connector 1000 is suitable for attachment to a syringe tip that does not include a radially outward extending flange thereon. Furthermore, an axial compressive force component as described above can be applied by flexing element 1048 or other biasing member(s) to assist in maintaining the mating or cooperating fittings in sealed engagement.

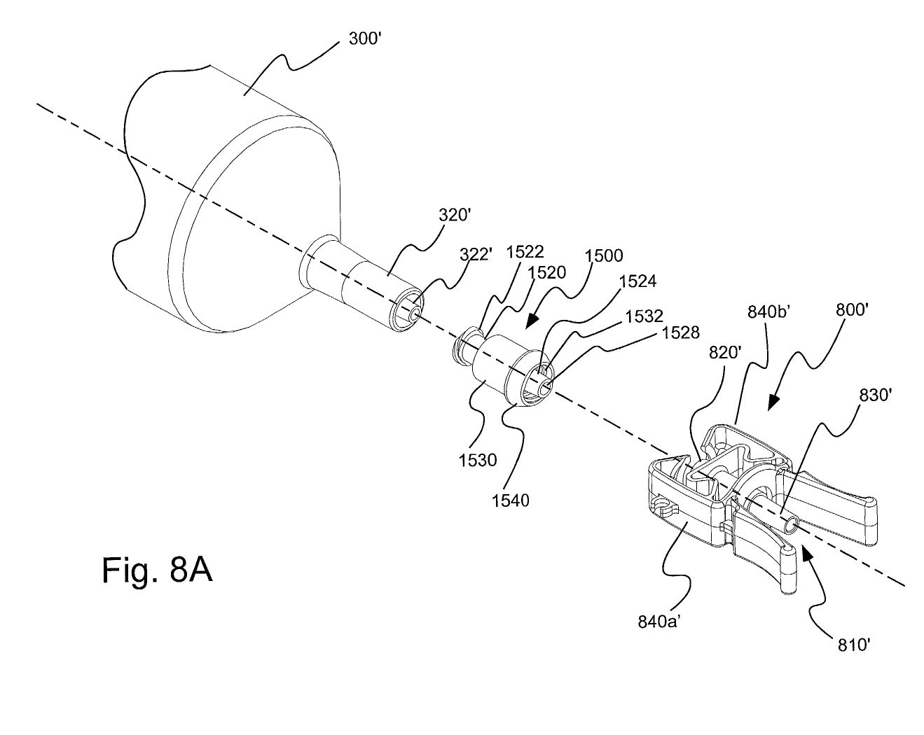

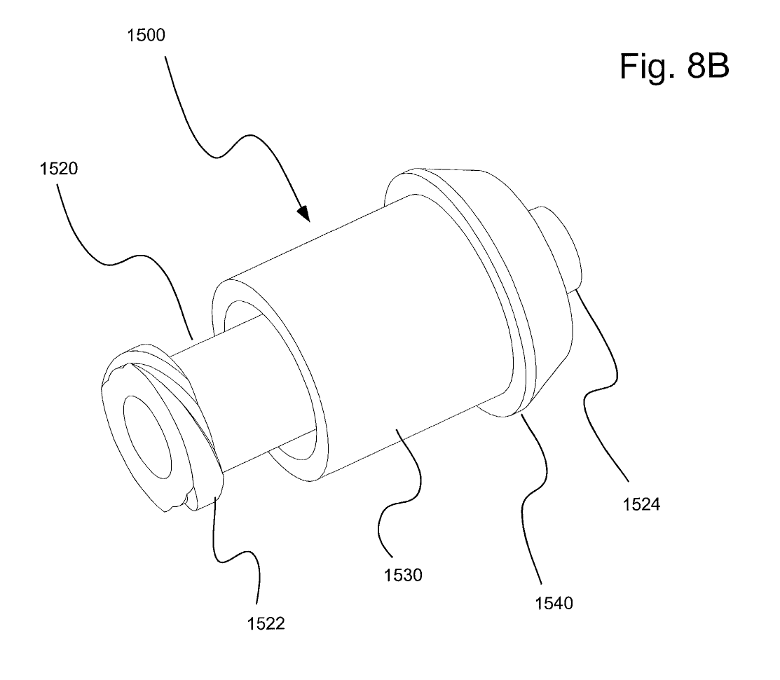

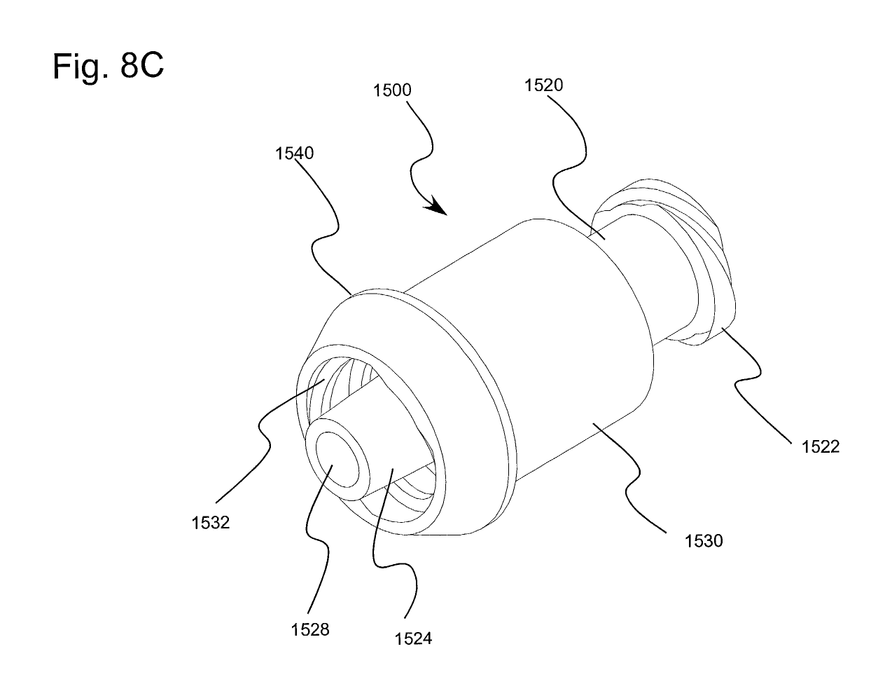

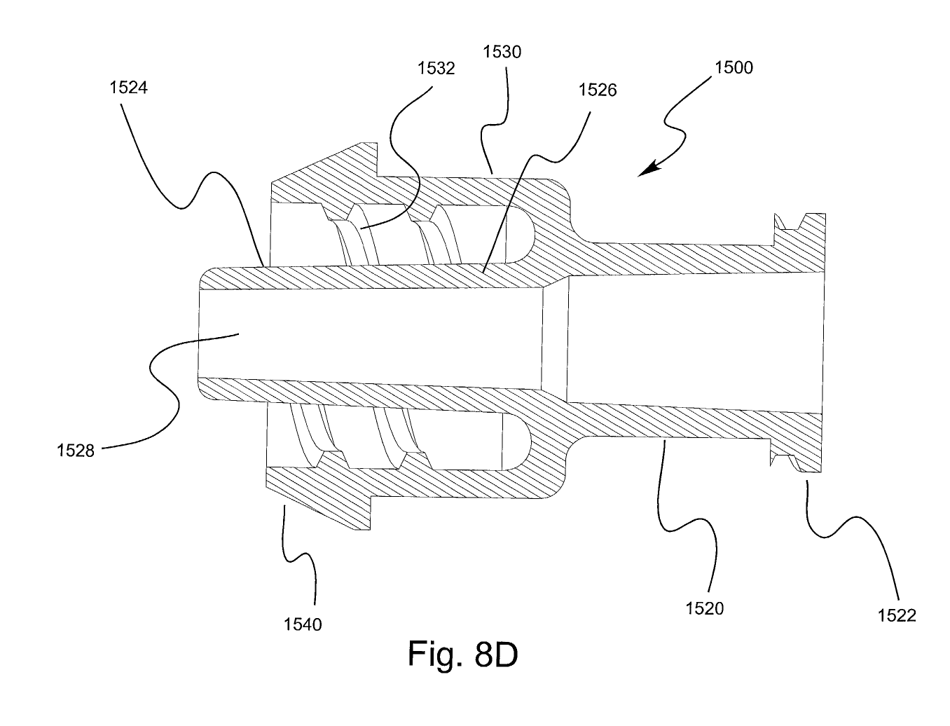

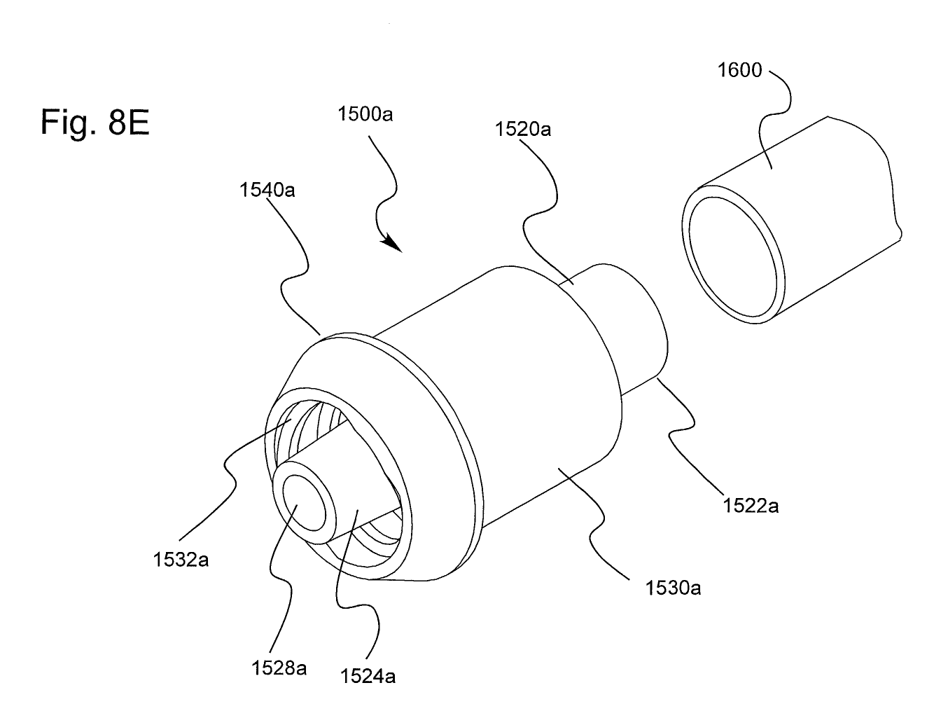

Indeed, many syringes and other articles to which it may be desirable to connect the connectors of the present invention do not include one or more attachment members or flanges such as attachment members 540, 540′ and 540″ described above. However, such attachment member can typically be incorporated into virtually any fluid path system using an adapter as illustrated in FIGS. 8A through 8D. In FIG. 8A a syringe 300′ having a syringe tip 320′ including a standard tapered male luer fitting 322′ (as described in connection with syringe 300) is illustrated. An adapter 1500 is preferably removably attachable to syringe tip 320 to provide an attachment member for cooperation with one or more of the connectors of the present invention.

In one embodiment, adapter 1500 includes a rear fitting or port that is, for example, a female luer attachment member 1520 on a rearward portion thereof including a radially outward extending helical flange 1522 that cooperates with threading (not shown) on syringe tip 320′ (as described, for example, in connection with luer connector 10 of FIG. 1) to removably connect adapter 1500 to syringe tip 320′. Adapter 1500 also includes a tapered male fitting 1524 on a forward portion thereof. As illustrated, for example, in FIG. 8D, female fitting 1520 and male fitting 1524 are formed integrally as a conduit member 1526 and are in fluid communication via passage 1528 formed in conduit member 1526.

A generally cylindrical surface, member or barrel 1530 is attached to conduit member 1526 to surround a portion of male tapered fitting 1524. An interior wall of barrel 1530 can include threading 1532 to, for example, cooperate with standard female luer fittings as described above. An attachment or flange member 1540 extends radially outward from barrel 1530. Attachment member 1540 operates in the same or a similar manner to attachment member 540′ described above. In that regard, a connector 800′ cooperates with attachment member 1540 as described above for connector 800 and attachment member 540′. Connector 800′ preferably includes a conduit member 810′ that includes a tapered female fitting 820′ on a rearward end thereof which is in fluid connection with a fitting or port 830′ having, for example, an exterior male taper (for example, a luer taper) on a forward end of conduit member 810′. Tubing can, for example, be solvent welded to the interior wall of fitting 830′. Moreover, a luer slip fit as known in the art can be formed between the tapered exterior wall of fitting 830′ and another fluid path element (for example, a valve). Fluid path connector 800′ also preferably includes a pair of rearward extending, resilient gripping arms 840a′ and 840b′ to form a connection with attachment member 540′ of syringe 500′ (or with, for example, attachment member 540 of syringe 500).

Adapter 1500 is preferably fabricated from a resilient, polymeric material such as a polycarbonate, polyethylene teraphthalate, ABS, or polyurethane via, for example, an injection molding process. Preferably, adapter 1500 is formed as an integral piece of such polymeric material via injection molding. Suitable polymeric materials, for example, preferably exhibit a flexural modulus and a tensile strength at yield as described above for connectors 700, 800 and 800′.

As illustrated in FIG. 8E, the adapters of the present invention can include rearward fittings other than female luer fittings to attach to virtually any type of fluid path component. In FIG. 8E, adapter 1500 includes a rear fitting that is designed to attach directly to a length of tubing 1600 using attachment methods known in the art. Other fluid path components, fittings or connector can be attached to the end of tubing 1600 opposite the end thereof attached to adapter 1500a. Virtually, any type of fitting as known in the art can be included as a rearward fitting in the adapters of the present invention (for example, female luer fittings, valves such a check valves, male luer fittings etc.).

As described above, adapter 1500a includes a tapered luer fitting 1524a such as a luer fitting on a forward portion thereof. A generally cylindrical surface 1530a surrounds male luer fitting 1524a. An interior wall of barrel 1530a includes threading 1532a as described above. An attachment or flange member 1540a extends radially outward from surface 1530a.

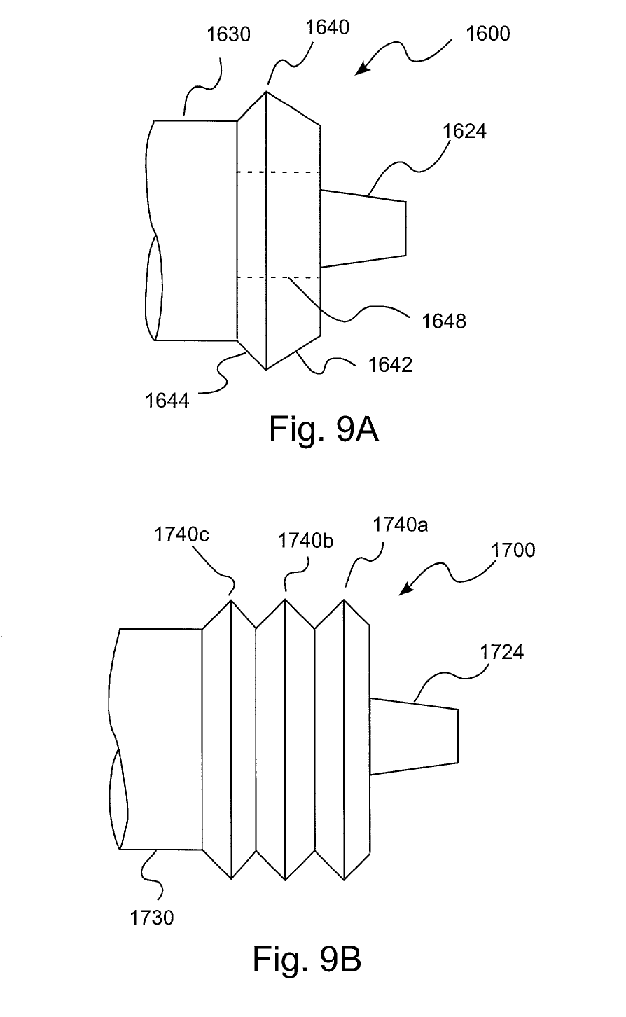

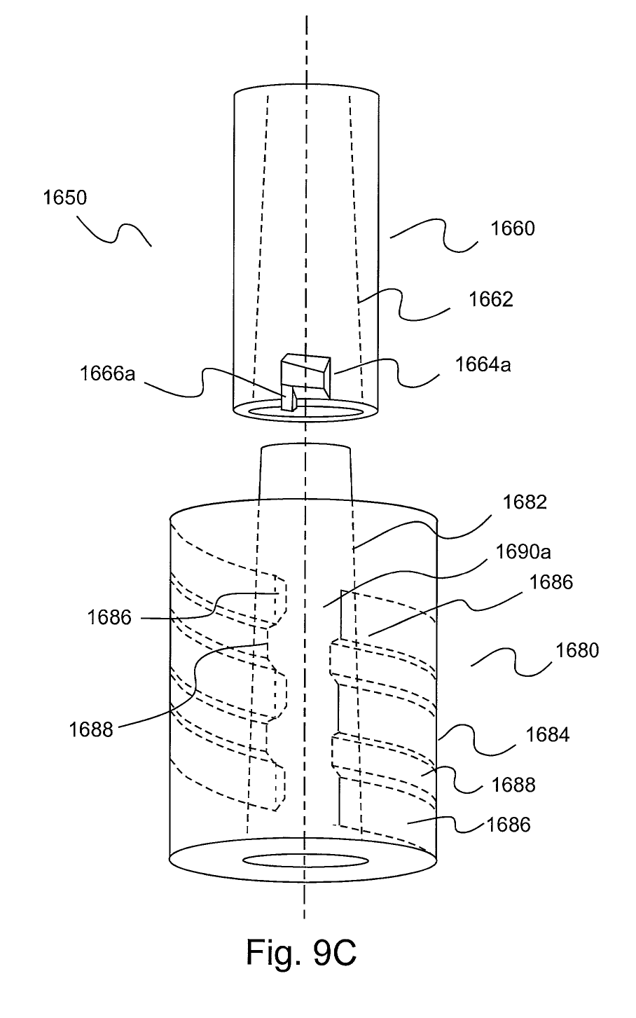

Although adapter 1500 and 1500a have been described as having a single circumferential attachment member or flange 1540 and 1540a, respectively, adapters and other fluid path connecting elements for use in the present invention can include other types of attachment members. For example, FIGS. 9A and 9B illustrate alternative connecting members 1600 and 1700 that can, for example, be formed on the end a syringe, adapter or other fluid path component. Connector 1600 includes a generally cylindrical surface, member or barrel 1630 to surround a portion of male tapered fitting 1624 as described above in connection with adapter 1500. An interior wall of barrel 1630 can include threading (not illustrated in FIG. 9A) to, for example, cooperate with standard female luer fittings as described above. An attachment or flange member 1640 extends radially outward from barrel 1630. Attachment member 1640 operates in the same or a similar manner to attachment member 540′ described above. However, attachment member 1640 includes a front, sloped surface 1642 that decreases in radius from back to front and a rear, sloped surface 1644 that increases in radius from back to front. Front, sloped surface 1642 can assist in forming a connection between, for example, resilient gripping arms 840a′ and 840b′ and attachment member 1600 as connector 800′ is brought into connection with connector 1600. Rear, sloped surface 1644 can assist in maintaining an axial compressive force between the mating or cooperating fittings of, for example, connector 800′ and connector 1600.

In the connectors described above, gripping arms have been flexed radially outward to allow passage of abutment flanges or gripping elements over an attachment member such as attachment member 1640. However, one or more openings or slots 1648 (illustrated in dashed lines in FIG. 9A) can be provided so that abutment flanges can pass therethrough without the necessity to flex the gripping arms. The connector can then be rotated (for example, ¼ turn) to bring the abutment flanges into abutting contact with attachment member 1640 in the manner of a bayonet connection. Rear sloped surface 1644 can be canted or pitched to increase the axial compressive force between the cooperating fittings upon rotation of the connector. Stop members and/or alignment members can be provided as known for bayonet connections to ensure that a proper connection has been made.

FIG. 9C illustrates another embodiment of a bayonet-type connector 1650 of the present invention. Connector 1650 includes a female section 1660 including a tapered female fitting or passage 1662 (shown in dashed lines in FIG. 9C) formed in the interior thereof. Female section 1660 also includes at least one radially outward projecting tab 1664a and, preferably, a pair of opposing tabs 1664a and 1664b (not shown in FIG. 9C, but generally identical to tab 1664a). Connector 1650 further includes a male section 1680 including a tapered male fitting 1682 to cooperate with female fitting 1662. Male section 1682 is surrounded by a generally cylindrical section or barrel 1684 having partially helical flanges 1686 (portions of several of which are illustrated in dashed lines in FIG. 9C) extending radially inward from an interior wall thereof. Partially helical flanges 1686 are formed generally parallel to or equidistant from each other and form partially helical channels 1688 therebetween. The phrase “partially helical” as used herein, in reference to flanges 1686 refers to the fact that flanges 1686 are canted or pitched and preferably follow the path of a helix but are not continuous around the inner circumference of barrel 1684. In general, partially helical flanges 1686 can be thought of or formed as a continuous helical or threaded flange with at least one axially oriented slot 1690a formed therethrough. Preferably, a pair of opposed slots 1690a and 1690b (not shown, but generally identical to slot 1690a) are formed therein.

During connection of female section 1660 and male section 1680, female section 1660 is move axially relative to male section 1680 so that tabs or projections 1664a and 1664b pass axially through slots 1690a and 1690b, respectively. Once female fitting 1662 is brought into contact with male fitting 1682, female section 1660 is rotated about its axis relative to male section 1680 so that tabs 1664a and 1664b turns through one of channels 1688 and is brought into abutting contact with one of flanges 1686. A stop member 1666a can, for example, be provided on tab or projection 1664a to stop rotation of female section 1660 by, for example, abutment with the end of one of flanges 1686 (defined, for example, by slot 1690). Unlike, current luer connectors in which a male section and a female section are rotated to form a sealed connection, approximately ¼ turn or 90° of rotation or less are required increase axial compression between female section 1660 and male section 1680 and to from a sealed connection between female section 1660 and male section 1680 that is suitable for use at low pressures or even pressures of 300 psi and above. Tabs 1664a and 1664b can be canted and generally formed in a shape to facilitate rotation thereof within one of channels 1688 as known in the threading arts.

Connector 1700 operates in a similar manner to connector 1600, except that connector 1700 includes a plurality of attachment members 1740a-c extending radially outward from barrel 1730, which surrounds a tapered male fitting 1724. Each of attachment members 1740a-c includes a front, sloped surface that decreases in radius from back to front and a rear, sloped surface that increases in radius from back to front, as described above for attachment member 1640. The plurality of attachment members 1740a-c can act as a ratchet mechanism for attachment of connectors such as connectors 800′ thereto and can reduce tolerance specifications on such connectors. As clear to one skilled in the art, fluid path connective elements of the present invention can be provided with many other types of attachment members.

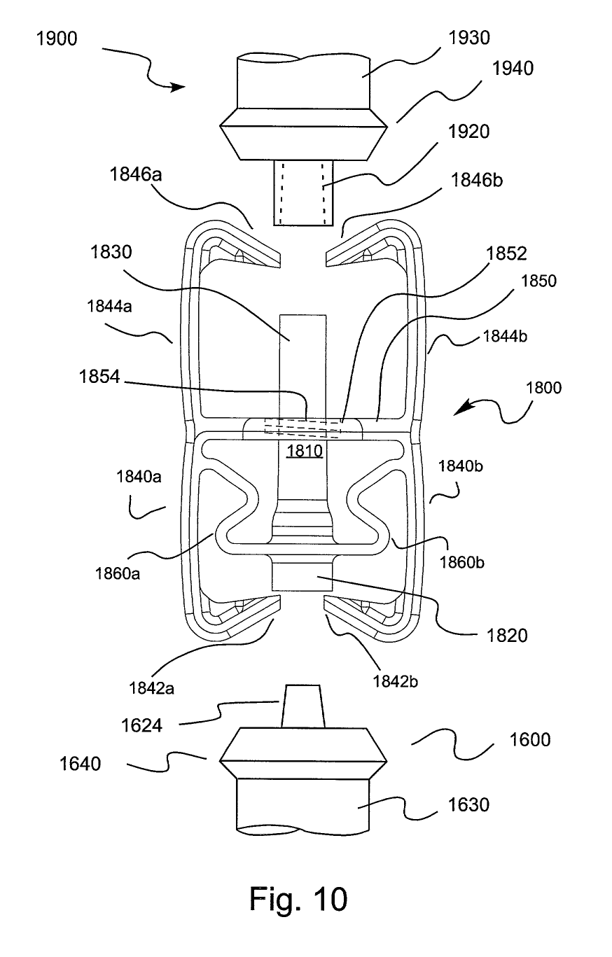

FIG. 10 illustrates another embodiment of a connector 1800 of the present invention. Connector 1800 can include a conduit member 1810 that includes a tapered female fitting 1820 (for example, a female luer fitting) on a first (for example, rearward) end thereof which is in fluid connection with a male fitting 1830 (for example, a male luer fitting) on a second (for example, forward) end thereof. Fluid path connector 1800 also preferably includes cooperating attachment members to form a connection with an attachment member such as attachment members 1640, 1740a etc. described above.

In that regard, connector 1800 includes a first pair of resilient gripping arms 1840a and 1840b , which terminate in gripping or abutment elements 1842a and 1842b, respectively. Gripping arms 1840a and 1840b are connected via a bridge 1850 as described above. Connector 1800 also includes spring arms 1860a and 1860b that operate to maintain an axial compressive force between mating fittings when connector 1800 is connected to another fluid path element. Connector 1800 further includes a second pair of resilient gripping arms 1844a and 1844b, which extend axially in the opposite direction of gripping arms 1840a and 1840b, and which terminate in gripping or abutment elements 1846a and 1846b, respectively.

Tubing can, for example, be solvent welded to the interior wall of fitting 1830 as described. As also described above, a luer slip fit as known in the art can be formed between the tapered exterior wall of fitting 1830 and another fluid path element. Resilient gripping arms 1844a and 1844b and gripping members 1846a and 1846b can form a connection with a connecting element 1900 of, for example, a syringe or other fluid path element.

In that regard, connecting element 1900 includes a tapered female fitting 1920 (for example, a female luer fitting) that forms a mating or cooperating connection with tapered male fitting 1830 of connector 1800. Connecting element 1900 also includes an attachment member such as a flange 1940 that extends radially outwardly from a barrel 1930 to cooperate with or form an abutting connection with gripping members 1846a and 1848b. Resilient spring arms 1860a and 1860b are caused to be extended toward connecting element 1600 when female fitting 1920 and male fitting 1830 are brought into engagement and gripping members 1846a and 1846b are brought into connective engagement with attachment member 1940. Spring arms 1860a and 1860b are prevented from returning to a non-stressed, resting or unextended state by contact between female fitting 1920 and male fitting 1830 and by abutment of gripping elements 1846a and 1846b with attachment member 1940. The resultant axial compressive force between female fitting 1920 and male fitting 1830 assists in maintaining a sealed connection therebetween even under significant fluid pressure.

In the embodiment of FIG. 10 spring elements 1860a and 1860b are used to assist in forming an axial compressive force between cooperating fittings for both female fitting 1820 and male fitting 1624. As clear to one skilled in the art, additional spring arms can be positioned, for example, between resilient gripping arms 1844a and 1844b and male fitting 1830 to assist in forming such an axial compressive force between mating or cooperating fittings.

Passage 1852 in bridge 1850 can also include threading 1854 to allow connection of male fitting 1830 to a standard female luer fitting such as fitting 170 as illustrated, for example, in FIG. 4D.

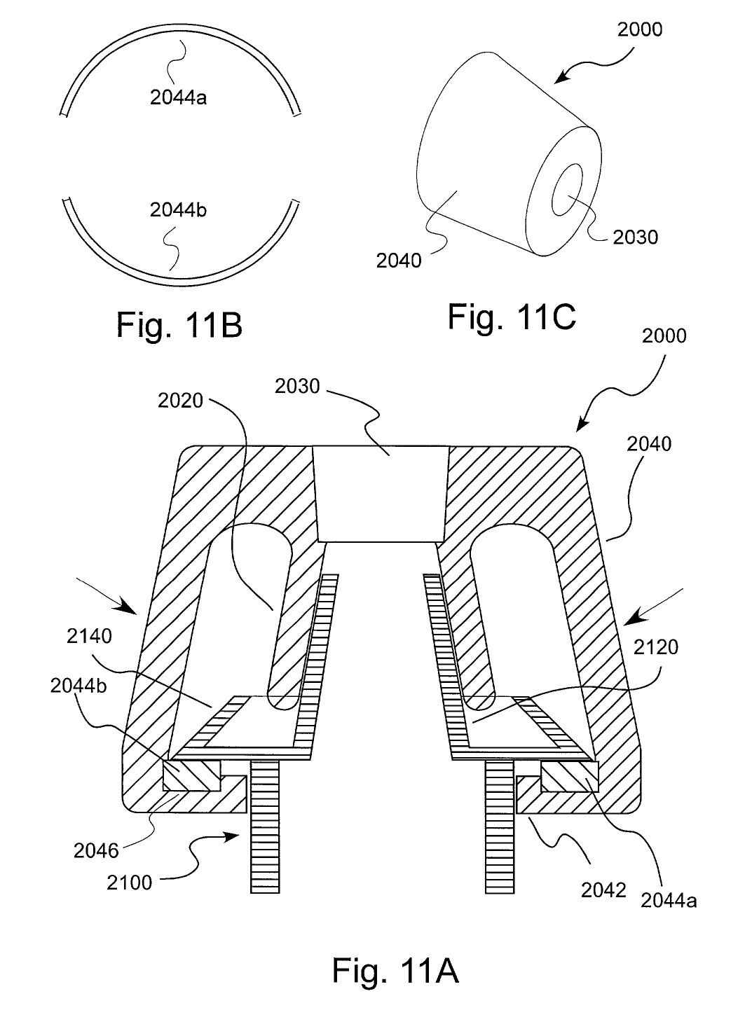

FIGS. 11A through 11C illustrate another embodiment of a connector 2000 of the present invention. Connector 2000 is illustrated connected to, for example, a connecting element 2100 of a fluid path element (for example, a syringe (not shown)) including a tapered male fitting 2120 and a radially outward extending flange 2140. In the embodiment of FIGS. 11A through 11C, connector 2000 includes a tapered female fitting 2020 in fluid communication with a port 2030 (for example, to which tubing or other fluid path elements can be attached). Connector 2000 also includes a generally frusto-conical gripping section 2040 which includes a radially inward extending gripping or abutment flange 2042 formed on a rearward end thereof. Female fitting 2020, port 2030, gripping section 2040 and abutment flange 2042 of connector 2000 can, for example, be fabricated from an integral piece of an elastomeric material (for example, polyurethane). Connector 2000 can also include one or more (two in the embodiment of FIGS. 11A through 11C) arced members 2044a and 2044b (which can, for example, be fabricated from a relatively inelastic, plastic material) seated in an annular channel or seating 2046 formed in abutment flange 2042 to assist in forming a secure abutting connection between attachment member 2140 and connector 2000.

A user can, for example, compress or squeeze in on the sides of gripping section 2040 (as represented by arrows in FIG. 11A) to cause abutment flange 2042 to flare open to assist in passing abutment flange 2042 over attachment member 2140 during connection to and/or removal from connecting element 2100. The resilient nature of connector 2000 and the dimensioning thereof maintains an axial compressive force between female fitting 2020 and male fitting 2120 during connection.

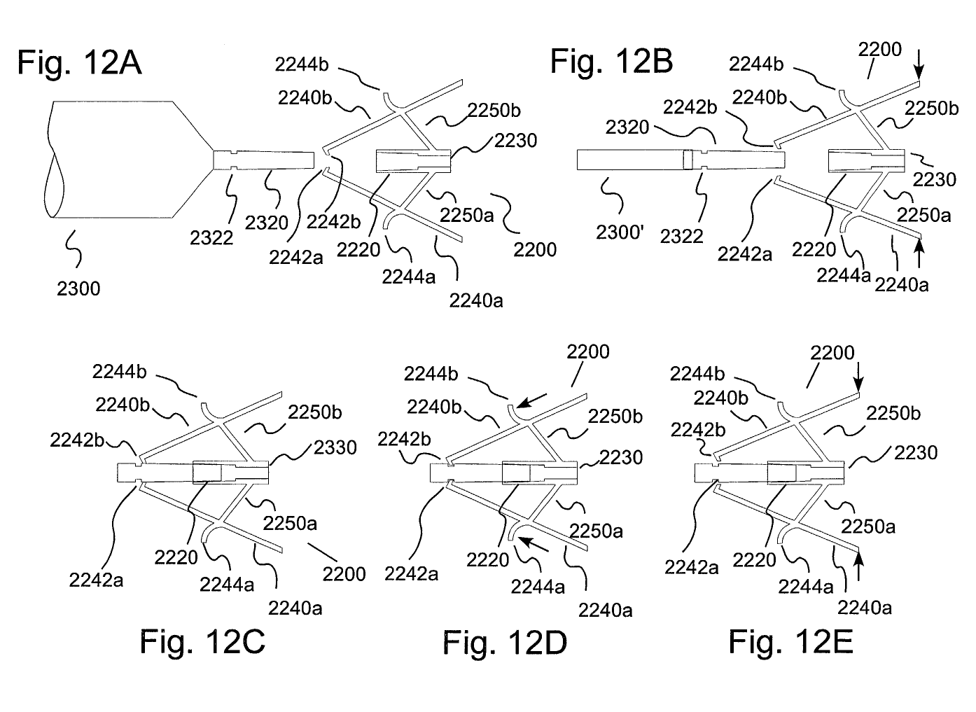

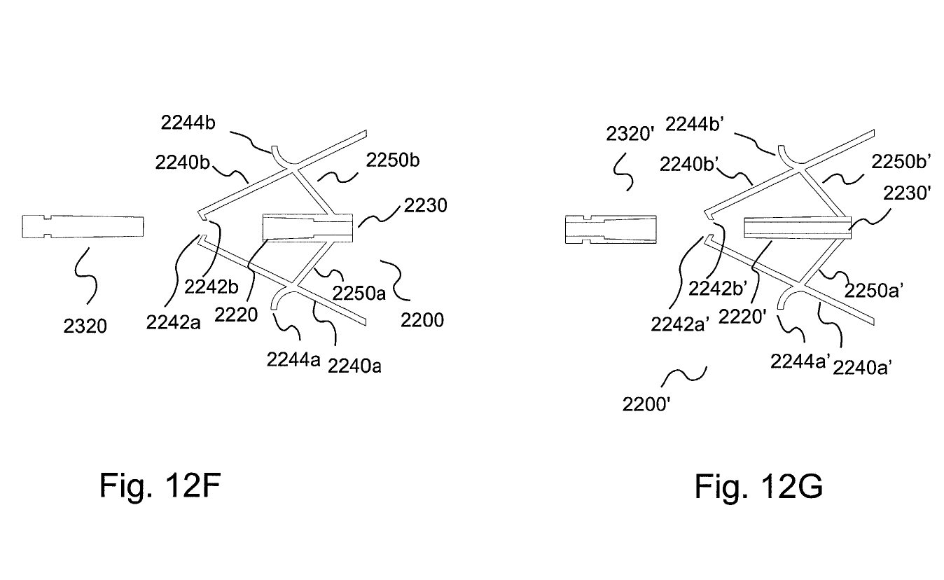

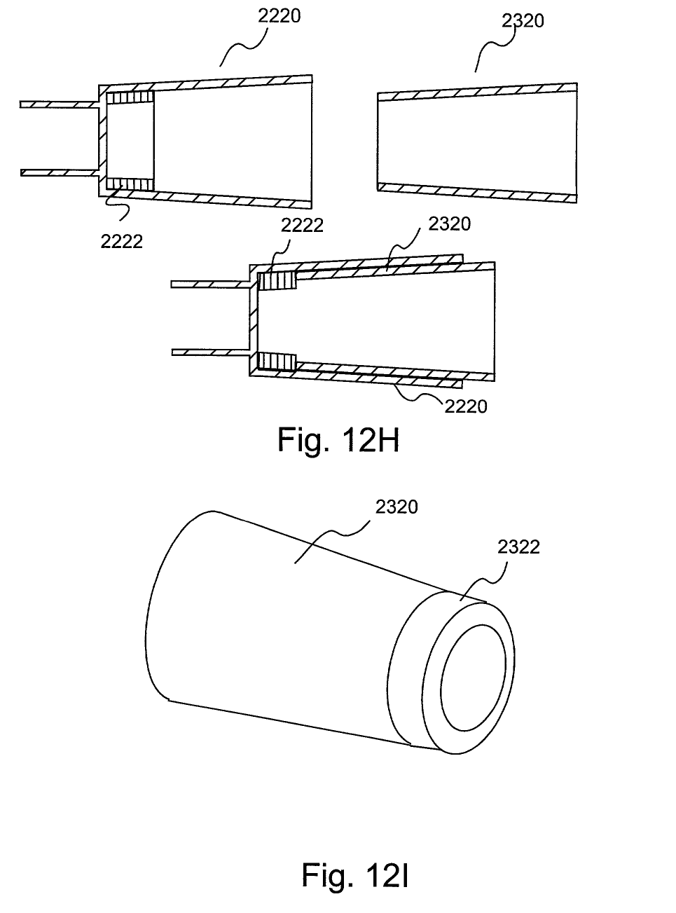

FIGS. 12A through 12G illustrate connector 2200 (FIGS. 12A through 12F) and connector 2200′ (FIG. 12G) of the present invention. FIGS. 12A and 12B illustrate alignment of connector 2200 to be attached to a tapered male fitting 2320 of a syringe 2300 and to a tapered male fitting 2320 of a tubing set 2300′. In that regard, connector 2200 includes a cooperating tapered female fitting 2220. Female fitting 2220 is in fluid connection with a port 2230. Female fitting 2220 is connected to resilient gripping arms 2240a and 2240b via bridging or connecting members 2250a and 2250b, respectively. Resilient gripping arms 2240a and 2240b include radially inward oriented gripping or abutment members or flanges 2242a and 2242b on a rearward end thereof. Resilient gripping arms 2240a and 2240b can also include radially outward extending contacts 2244a and 2244b, respectively, the operation of which is described below.

During connection of connector 2200 to male fitting 2320, the user can first compress gripping arms 2240a and 2240b radially inward at a forward end thereof as represented by arrows in FIG. 12B to cause the rearward end of gripping arms 2240a and 2240b and abutment flanges 2242a and 2242b to move radially outward. Connector 2200 and male fitting 2320 are then move axially together. A rearward surface of abutment flanges 2242a and 2242b can be sloped to facilitate connection. Female fitting 2220 and male fitting 2320 are brought into contact as in a standard interference or friction fit as illustrated in FIG. 12C. At this point, the user can push radially inward/forward on contacts 2244a and 2244b as represented by arrows in FIG. 12D to cause abutment flanges 2242a and 2242b to move forward to seat in annular channel 2322 formed in male fitting 2320. Abutment of abutment flanges 2242a and 2242b with the forward surface of annular channel 2322, prevent gripping arms 2240a and 2240b and bridge members 2250a and 2250b from returning to their unstressed or rest state (see, for example, FIG. 12B). The resilient nature of connector 2200 thereby results in an axial compressive force between female fitting 2220 and male fitting 2320 when abutment flanges 2242a and 2242b are seated in annular channel 2322. To remove connector 2200 from male fitting 2320, the user can compress gripping arms 2240a and 2240b (as represented by arrows in FIG. 12E) to cause abutment flanges 2242a and 2242b to unseat from annular channel 2322 and move forward 15 toward their rest state. Connector 2200 can then be move axially away from male fitting 2300 to complete the disconnection.

In general, each connector of the present invention discussed above has be described with a female fitting thereon which is connected to a male fitting on another fluid path element (for example, a syringe). As clear to one skilled in the art, however, the connectors of the present invention can include a male fitting for connection to a female fitting on another fluid path element. For comparison, FIGS. 12F and 12G, respectively illustrate connector 2200 including a female fitting 2220 aligned to be connected male fitting 2320 and a connector 2200′ including a male fitting 2220′ aligned to be connected to a female fitting 2320′. In other respects, connector 2200′ is identical to connector 2200 and 2 5 corresponding elements thereof are numbered similarly with the addition of a “′” designation.

FIGS. 12H and 12I illustrates the use of compressible sealing rings to enhance the sealing connection between the cooperating fitting of the connectors of the present invention. FIG. 12H illustrates the use of a compressible sealing member 2222 (for example, an elastomeric ring) disposed within female fitting 2220. The axial compressive force maintained between male fitting 2320 and female fitting 2220 during connection of male fitting 2320 to female fitting 2220 results in compression of sealing member 2222, which assists in forming a sealed connection therebetween. As clear to one skilled in the art, such a sealing member can be positioned on either cooperating fitting or on both fittings. For example, FIG. 12I illustrates a compressible annular sealing member 2322 attached to the end of male fitting 2320.

In the above embodiments, an axial compressive force assists in maintaining a sealing engagement between cooperating fittings. When used in connection with tapered (for example, luer) fittings, the above-described connectors are suitable for use at relatively higher pressures generated in powered injection procedures. Yet, unlike standard luer connectors, which typically require threaded connection for use at relatively high pressure, the connectors of the present invention can be connected to a cooperating fitting without rotation, using only one hand and without risk of breakage. In general, in maintaining a sealed connection at pressures of approximately 300 psi between a male luer fitting and cooperating female luer fitting (having approximately 6% tapers) in which the connector is fabricated from polycarbonate, and the cooperating fitting is fabricated from polyethylene teraphthalate, an axial compressive load/force of approximately 6-14 pounds was maintained.

The present inventors have also discovered that a friction fit between tapered (for example, luer) fittings can withstand relatively high internal fluid pressures (for example, in excess of 300 psi) even without maintaining an axial compressive force therebetween if the fittings are brought together/connected with a sufficient axial load. For example, the present inventors have discovered that an axial connecting load of approximately 10 to 14 pounds in the case of cooperating luer fitting of the materials described above is suitable for from a sealed connection under fluid pressures in excess of 300 psi.

Of course, the required axial load can change with, for example, different fabrication materials and different tapers. For example, more compliant materials may result in a lower required load. Also, physical characteristics that increase the coefficient of friction between the fittings (for example, via material choice or knurling) can also decrease the required load.

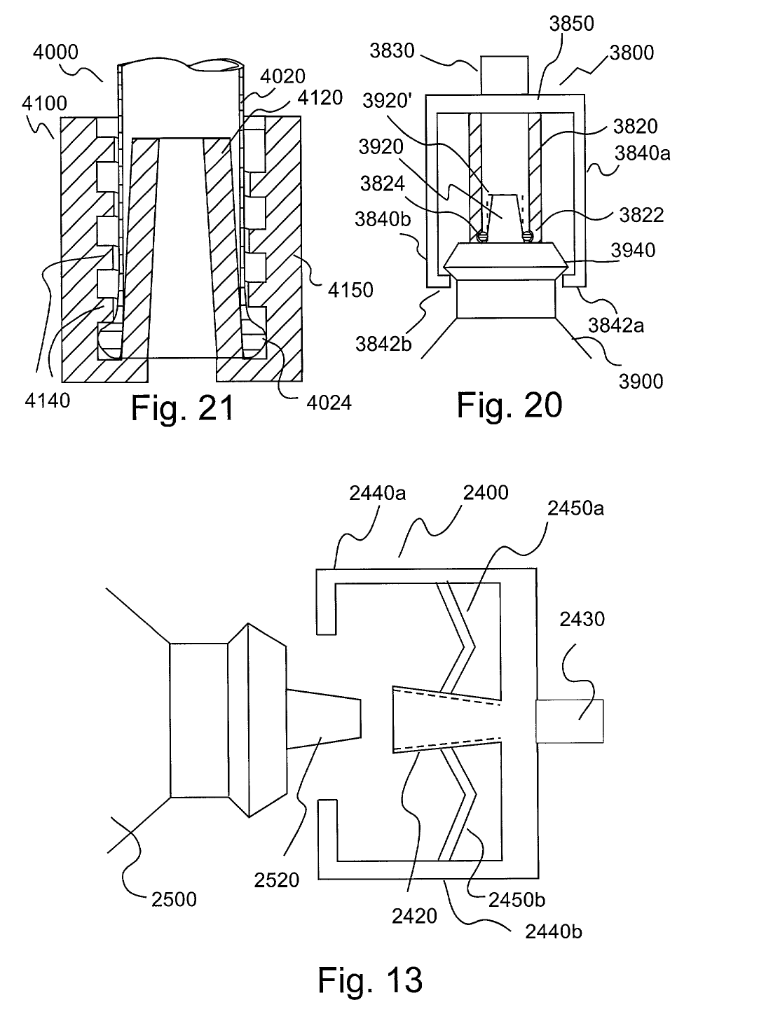

An indicator can be provided to give the user an indication that the fittings have been brought together with sufficient force to provide a sealed connection under pressure. In that regard, FIG. 13 illustrates a connector 2400 including a tapered female fitting 2420 in fluid connection with a port 2430. Connector 2400 is illustrated in general alignment for connection to a tapered male fitting 2520 of a syringe 2500. Connector 2400 also includes indicators 2450a and 2450b connected between side members 2440a and 2240b and female fitting 2420.

Indicators 2450a and 2450b can, for example, be fabricated from a polymeric material that permanently deforms when the desired axial load has been achieved. A visual, tactile and/or audible indication can be provided to the user. As clear to one skilled in the art, other types of indicators can be used in the connectors of the present invention.

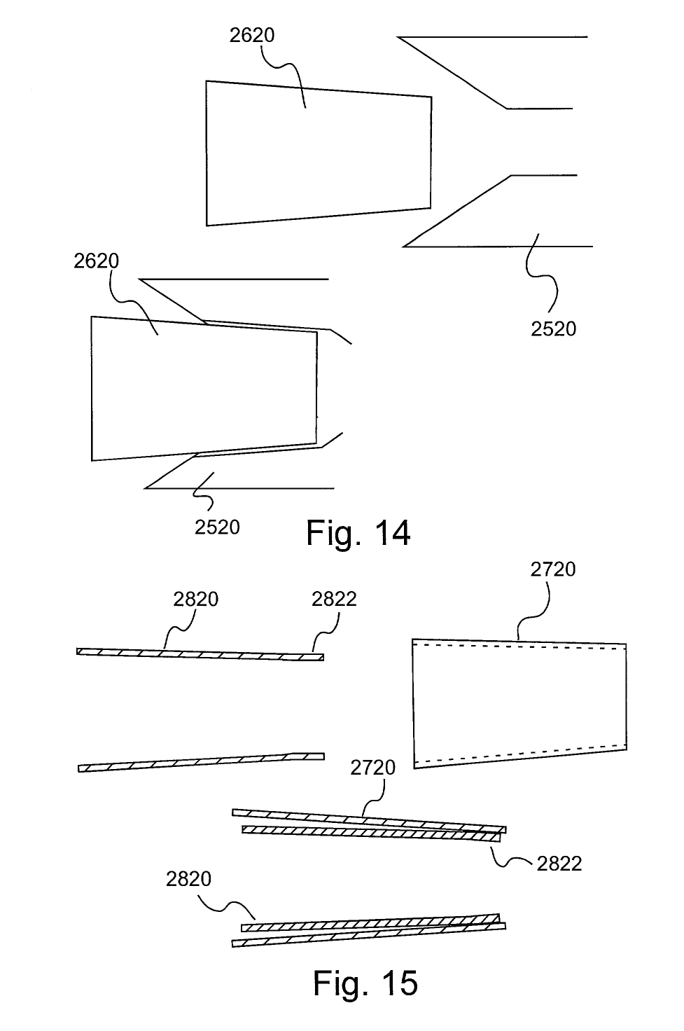

In addition to or even in the alternative to the axial loads used in the connectors of the present invention (whether maintained during connection or applied only upon connection), a radial sealing component can be applied in the connectors of the present invention to improve sealing. For example, FIGS. 14 and 15 illustrate the use of a compliant or resilient material (for example, polyurethane) on one of the cooperating fittings to improve the seal therebetween. In FIG. 14, a female fitting 2520 has a taper (for example, 12%) that is greater than the taper (for example, 6%) of a cooperating male fitting 2620. At least a portion of female fitting 2520 is fabricated from or coated with a resilient or compliant material such that contact of male fitting 2620 (for example, fabricated from a less resilient or harder material such as polycarbonate) with female fitting 2520 causes female fitting 2520 to deform to generally the shape of male fitting 2620.

FIG. 15 illustrates the case in which a male fitting 2820 includes a compliant or resilient portion 2822 on a front end thereof that deflects or deforms when contacted with a female fitting 2720. Deflecting portion or section 2822 can, for example, have a taper that is less than the remainder of male fitting 2820 (including the cases that portion 2822 has no taper or tapers in the opposite direction from the remainder of male fitting 2820. Deflecting portion 2822 can, for example, be fabricated from a material that is more compliant than the remainder of male fitting 2820 or can be fabricated from the same material but with a wall thickness that is less than the remainder of male fitting 2820.

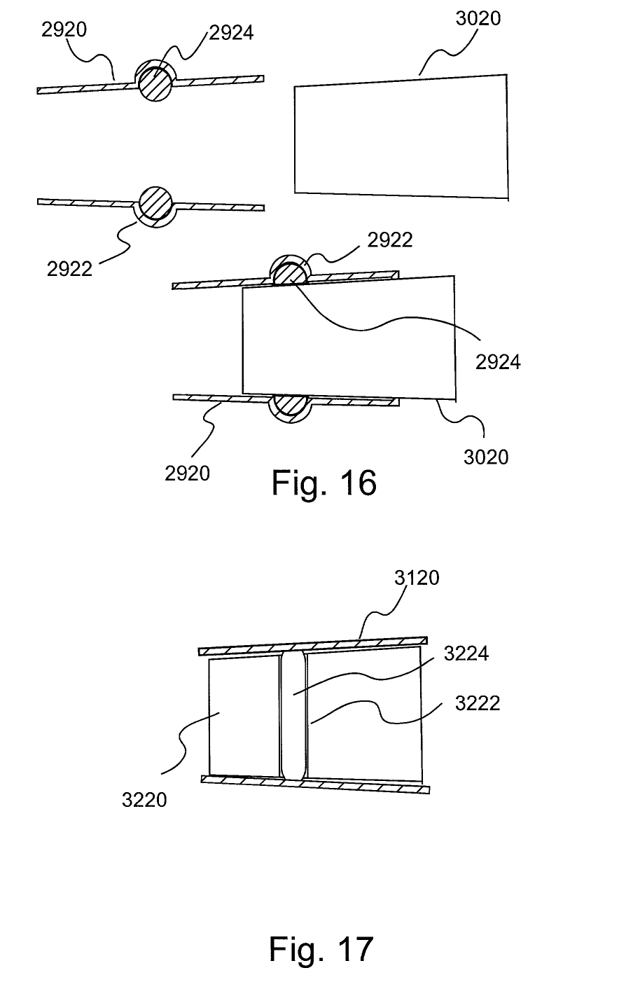

FIG. 16 illustrates a sealing member 2924 (for example, fabricated from an elastomeric material) seated in a channel 2922 formed in a tapered female fitting 2920. Sealing member 2924 is deformed by male fitting 3020 when male fitting 3020 is brought into connection with female fitting 2920 to form a seal between male fitting 3020 and female fitting 2920.

FIG. 17 illustrates a male fitting 3220 including an annular sealing member or seal 3224 positioned within a channel 3222 formed therein. Connection of male fitting 3220 with female fitting 3120 causes deformation of sealing member 3224 and forms seal between male fitting 3220 and female fitting 3120.

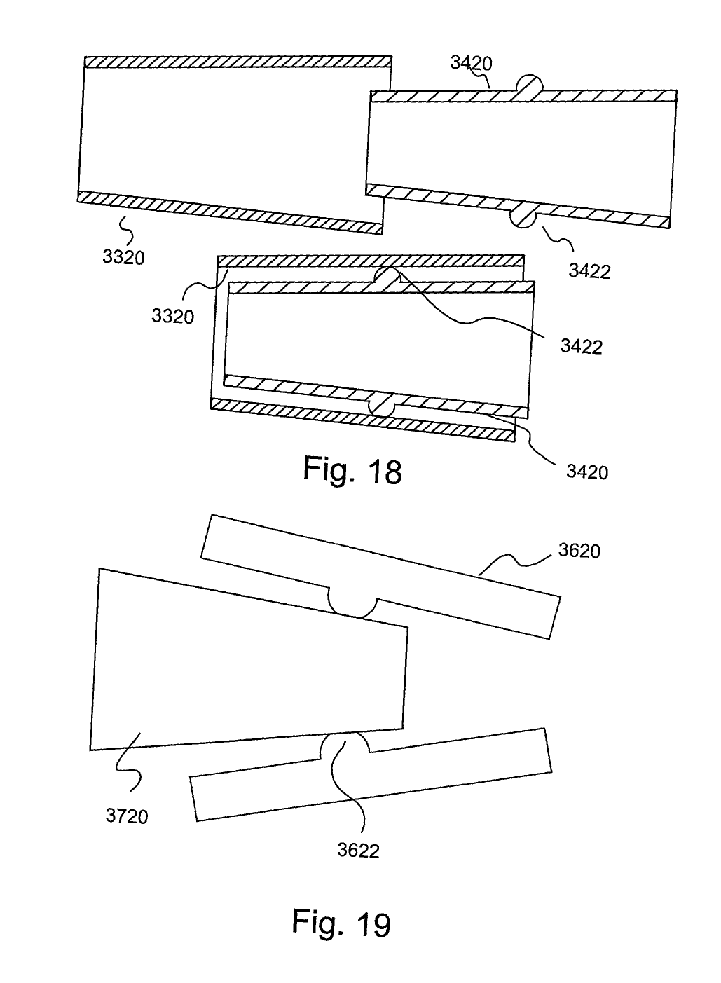

FIG. 18 illustrates a female fitting 3320 and a cooperating male fitting 3420 in which the male fitting 3420 includes a radially outward annular “crush” ring formed around the circumference thereof. Connection of female fitting 3320 and male fitting 3420 causes deformation or crushing of annular ring 3422 and forms a seal between female fitting 3320 and male fitting 3420.

FIG. 19 illustrates a female fitting 3520 including a radially inward annular ring 3622 that deforms upon connection with a cooperating male fitting 3720.

In the case that a radial sealing component or force is used in connectors of the present invention, it is possible in certain circumstances to maintain an adequate seal under pressure without maintaining an axial compressive force between the cooperating fittings. FIG. 20 illustrates a connector 3800 including a cooperating fitting 3820. Fitting 3820 need not be but can be tapered. Cooperating fitting 3820 includes a channel 3822 formed therein in which an annular sealing member 3824 (for example, an elastomeric ring) is seated. Fitting 3820 is in fluid communication with a port 3830 as described above in connection with other connectors of the present invention. Connector 3830 also includes axially extending gripping arms 3840a and 3840b, which include radially inward extending abutment flanges 3842a and 3842b, respectively, on a rearward end thereof. Extending arms 3840a and 3840b are connected to fitting 3820 via a bridge 3850.

Connector 3800 is illustrated in connection with a syringe 3900 including a cooperating fitting that can be a male tapered fitting 3920 or a non-tapered fitting 3920′ (shown in dashed lines). During connection of connector 3800 to syringe 3900, extending arms 3940a and 3940b flex radially outward to allow abutment flanges 3842a and 3842b to pass over an attachment flange 3940 of syringe 3900 to form an abutting connection therewith. Deformation of sealing member or seal 3824 between cooperating fitting 3820 of connector 3800 and cooperating fitting 3920 or 3920′ of syringe 3900 forms a sealed connection therebetween. Preferably, fittings 3820 and fitting 3920 or 3920′ as well as seal 3824 are dimensioned to allow for sealing connection over a range of relative axial positions between connector 3800 and syringe 3900 so that tolerance specifications for syringe 3800 and connector 3900 can be loosened. Like many of the connectors described above, connector 3800 can be molded from an integral piece of polymeric material to which seal 3824 can be added.

FIG. 21 illustrates another embodiment of a connector 4000 of the present invention which includes a length of deformable tubing 4020 with a radially outward extending flange 4024 formed on a rearward end thereof. Connector 4000 is shown in connection with a standard male luer connector 4100, which includes a male luer fitting 4120 and helical threading 4140 projecting radially inward from a barrel section 4150 as described above. Flange 4024 of tubing 4020 forms a sealed engagement with the inner wall of barrel 4150, while abutment of flange 4024 of tubing 4020 with threading 4140 (or other radially inward projecting flange(s)) prevents separation of connector 4000 from connector 4100 even under pressure. Preferably, flange 4024 passes axially to the rear of threading 4140 when connector 4000 is in connection with connector 4100 to allow flange 4024 to form a sealed connection with a generally cylindrical portion of the inner wall of barrel 4150.

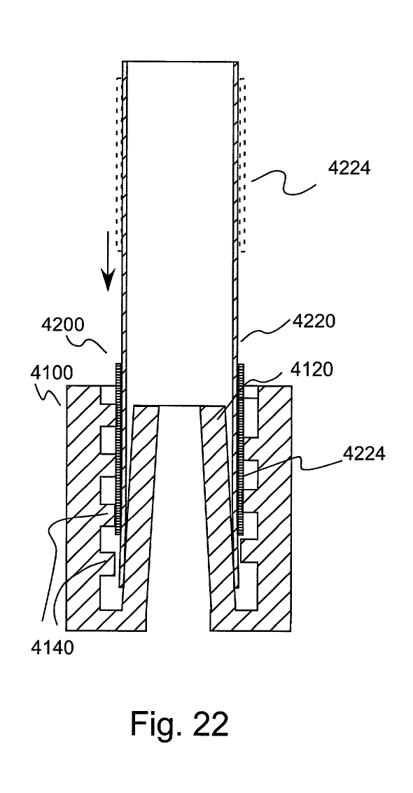

FIG. 22 illustrates a connector 4200 including a length of deformable tubing 4220 and a collar or collet 4224, which is slidably disposed on tubing 4220. During connection of connector 4200 to connector 4100, tubing 4220 is first slid over male fitting 4120. Collet 4224 is then slid rearward to be disposed between threading 4140 and tubing 4220 as illustrated in FIG. 22, thereby creating a radial compressive force and sealed connection between male fitting 4120 and tubing 4220.

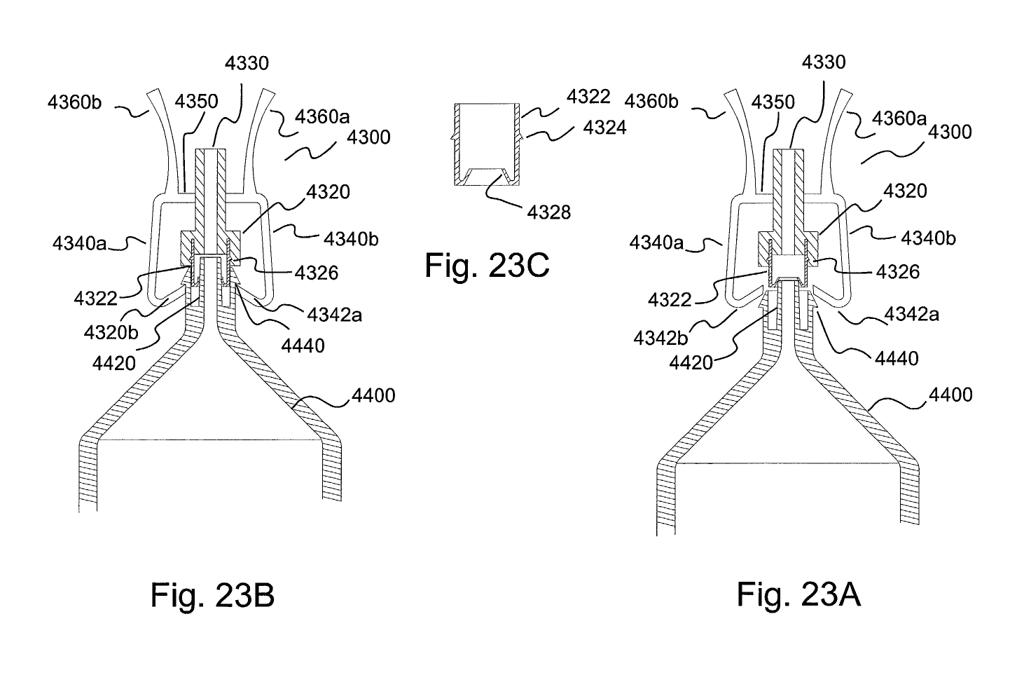

The connectors of the present invention can also include a “self-sealing” feature as illustrated in FIGS. 23A through 23C. Connector 4300 includes a female fitting 4320 in fluid connection with a port 4330. Female fitting 4320 includes a seal 4322 disposed therein via seating of a radially outward extending flange 4324 (see FIG. 23C) on seal 4322 within an annular channel 4326 formed in an inner wall of female fitting 4320. Connector 4300 further includes axially extending arms 4340a and 4340b having radially inward extending abutment flanges 4342a and 4342b , respectively, which cooperate with a flange 4440 on a syringe 4400 as described above, to connect connector 4300 to syringe 4400. Arms 4340a and 4340b can be connected via a bridge 4350. Connector can further include lever arms 4360a and 4360b to facilitate radially outward movement of arms 4340a and 4340b during connection and disconnection as described above.

During connection of connector 4300 to syringe 4400, a radially inward projecting flange 4328 formed on a rearward end of seal 4322 makes a sealed connection with a male fitting 4420 (for example, a tapered or luer male fitting) of syringe 4400. As fluid pressure increases within the system, the compressive sealing force between flange 4328 (which is in fluid contact with the pressurized fluid) and male fitting 4420 is increased. Abutment of flanges 4342a and 4342b cooperating flange 4440 on a syringe 4400, prevents axial disconnection of connector 4300 from syringe 4440.

Although the present invention has been described in detail in connection with the above examples, it is to be understood that such detail is solely for that purpose and that variations can be made by those skilled in the art without departing from the spirit of the invention. The scope of the invention is indicated by the following claims rather than by the foregoing description. All changes to the present invention that fall within the meaning and range of equivalency of the claims are to be embraced within their scope.

What is claimed is:

1. A syringe for use in a medical injection procedure in connection with a connector having a tapered fitting, the syringe comprising:
a syringe tip comprising a cooperating tapered fitting having a passage therein in fluid communication with an interior of the syringe;
a first connecting member comprising a radially inward threaded portion on an inner surface thereof adapted to connect to a first type of connector comprising a tapered fitting and having at least one radially outward extending flange to connect to the radially inward threaded portion of the first connecting member; and
a second connecting member comprising at least one abutment member to connect to a second type of connector comprising a cooperating tapered fitting and a cooperating attachment member comprising at least one radially inward projecting flange to engage the at least one abutment member of the second connecting member.

2. The syringe of claim 1 wherein the second connecting member comprises a threaded flange.

3. The syringe of claim 1 wherein the first connecting member is positioned on a radial interior side of a generally cylindrical member surrounding the cooperating tapered fitting and the second connecting member is positioned on a radial exterior side of the generally cylindrical member.

4. The syringe of claim 3 wherein the cooperating tapered fitting is a male luer fitting.

5. The syringe of claim 4 wherein the second connecting member comprises at least one radially outward extending flange.

6. The syringe of claim 4 wherein the second connecting member comprises at least one radially inward extending channel.

7. The syringe of claim 3 wherein the second connecting member comprises a plurality of radially outward extending flanges.

8. A syringe and connector system for use in a medical injection procedure, the syringe and connector system comprising:
a syringe comprising a syringe tip on a forward end of the syringe, the syringe tip comprising a tapered fitting having a passage therein in fluid communication with an interior of the syringe, the syringe further comprising a first connecting member comprising at least one abutment member to connect to a connector comprising a cooperating tapered fitting and a cooperating attachment member comprising at least one radially inward projecting flange to engage the at least one abutment member of the first connecting member, the syringe further comprising a second connecting member having a radially inward threaded portion on an inner surface thereof adapted to connect to a tapered connector having a cooperating tapered fitting and at least one radially outward extending flange adapted to connect to the radially inward threaded portion of the second connecting member; and
a connector comprising a cooperating tapered fitting, a cooperating attachment member comprising at least one radially inward projecting flange to engage the abutment member of the first connecting member, and at least one port in fluid connection with the cooperating tapered fitting.

9. The syringe and connector system of claim 8 wherein the abutment member comprises a single radially outward extending flange oriented generally perpendicular to an axis of the syringe tip.

10. The syringe and connector system of claim 8 wherein the abutment member comprises a single radially inward extending channel oriented generally perpendicular to an axis of the syringe tip.

11. The syringe and connector system of claim 8 wherein the abatement member comprises radially outward extending threading and the radially inward projecting flange of the connector comprises a threaded portion to engage the threading of the attachment member.

12. The syringe and connector system of claim 11 wherein the threaded portion of the connector is rotatable relative to the cooperating tapered fitting.

13. The syringe and connector system of claim 8 wherein the cooperating attachment member of the connector comprises at least one extending arm comprising at least one radially inward extending flange.

14. The syringe and connector system of claim 8 wherein the cooperating attachment member of the connector comprises a plurality of extending arms, each of the arms comprising at least one radially inward extending flange.

15. The syringe and connector system of claim 14 wherein the radially inward extending flanges of the arms are biased radially inward when the connector and the syringe are engaged.

16. The syringe and connector system of claim 15 wherein the arms are resilient, flexing arms and are biased radially inward by a bending moment.

17. The syringe and connector system of claim 15 wherein the arms are biased radially inward by a locking member attached to the connector.

18. The syringe and connector system of claim 14 wherein the connector further comprises a biasing member to provide axially oriented force directed to retain the tapered fitting of the syringe and the cooperating tapered fitting of the connector in sealing engagement.

19. The syringe and connector system of claim 18 wherein the biasing member comprises at least one resilient spring arm in operative connection with the cooperating tapered fitting.

20. The syringe and connector system of claim 18 wherein the biasing member comprises a plurality of resilient spring arms in operative connection with the cooperating tapered fitting.

21. The syringe and connector system of claim 11 wherein the cooperating attachment member of the connector comprises a plurality of resilient, extending arms, each of the arms comprising a radially inward extending flange having at least one groove formed thereon to mate with the threading of the attachment member.

22. The syringe and connector system of claim 21 wherein the arms are biased radially inward by a bending moment when the syringe and the connector are connected.

23. The syringe and connector system of claim 22 wherein the connector is rotatable relative to the syringe after connection thereto to provide axially oriented force directed to retain the tapered fitting of the syringe and the cooperating tapered fitting of the connector in sealing engagement.

24. The syringe and connector system of claim 23 wherein the arms prevent over tightening during rotation of the connector by flexing radial outward during rotation to slip over the threading of the attachment member.

25. The syringe and connector system of claim 8 wherein the cooperating attachment member of the connector comprises at least one radially inward extending flange having an adjustable angle of orientation, the flange having a locking orientation in which it resists disengagement from the abutment member and a disengagement orientation in which the flange can pass over the abutment member of the syringe to be placed in engagement with or to be removed from engagement with the abutment member of the syringe.

26. The syringe and connector system of claim 25 wherein the locking orientation forms a first angle with respect to the axis of the syringe tip, and the disengagement orientation forms a second angle with respect to the axis of the syringe tip, the second angle being closer to perpendicular to the axis of the syringe tip than the first angle.

27. The syringe and connector system of claim 8 further comprising a deformable seal to form a seal between the tapered fitting and the cooperating tapered fitting.

28. A connector for use with a tapered fitting assembly having at least one attachment member, the connector comprising:
a cooperating tapered fitting;
at least one port in fluid connection with the cooperating tapered fitting; and
a cooperating attachment member attached to the cooperating fitting to engage the attachment member of the tapered fitting assembly, a predetermined level of force being require to cause the cooperating attaching elements to form a cooperating connection with the at least one attachment member of the tapered fitting assembly.

29. The connector of claim 28 wherein the cooperating attachment member engages the attachment member via relative axial motion of the cooperating tapered fitting and the fitting assembly.

30. The connector of claim 28 wherein the cooperating attachment member engages the attachment member via relative axial motion of the cooperating attachment member and the fitting assembly followed by rotation of the cooperating attachment member relative to the fitting assembly.

31. The connector of claim 30 wherein the cooperating attachment is rotated no more than 90°.

32. The connector of claim 28 wherein the cooperating attachment member comprises at least one axially extending arm having at least one radially inward extending flange projecting therefrom.

33. The connector of claim 28 wherein the cooperating attachment member comprises a plurality of extending arms, each of the arms having at least one radially inward extending flange projecting therefrom

34. The connector of claim 33 the flanges of the arms are biased radially inward in connection with the attachment member of the tapered fitting assembly when the connector and the fitting assembly are engaged.

35. The connector of claim 34 wherein the arms are resilient, flexing arms and are biased radially inward by a bending moment.

36. The connector of claim 34 wherein the arms are biased radially inward by a locking member attached to the connector.

37. The connector of claim 33 wherein the connector further comprises a biasing member to provide axially oriented force directed to retain the tapered fitting assembly and the cooperating tapered fitting of the connector in sealing engagement.

38. The connector of claim 37 wherein the biasing member comprises at least one resilient spring arm in operating connection with the cooperating tapered fitting.

39. The connector of claim 37 wherein the biasing member comprises a plurality of resilient spring arms in operating connection with the cooperating tapered fitting.

40. The connector of claim 33 wherein each of the plurality extending arms is flexible and resilient and each of the radially inward extending flanges of the extending arms comprises at least one groove to mate with threading of the attachment member of the tapered fitting assembly.

41. The connector of claim 40 wherein the arms are biased radially inward by a bending moment when the syringe and the connector are connected.

42. The connector of claim 41 wherein the connector is rotatable relative to the tapered fitting assembly after connection thereto to provide axially oriented force directed to retain the tapered fitting assembly and the cooperating tapered fitting of the connector in sealing engagement.

43. The connector of claim 42 wherein the arms prevent over tightening during rotation of the connector by flexing radial outward during rotation to slip over the threading of the attachment member.

44. A connector for use with a tapered fitting assembly, the connector comprising:
a cooperating tapered fitting;
a cooperating attachment member in operative attachment with the cooperating fitting to engage the tapered fitting assembly; and
at least one port in fluid connection with the cooperating tapered fitting; the cooperating attachment member comprising a radially inwardly extending flange having an adjustable angle of orientation, the flange having a locking orientation in which it resists disengagement from the tapered fitting assembly and a disengagement orientation in which the flange can be placed in engagement with the tapered fitting assembly or be removed from engagement with the tapered fitting assembly.

45. The connector of claim 44 wherein the locking orientation forms a first angle with respect to the axis of the syringe tip, and the disengagement orientation forms a second angle with respect to the axis of the syringe tip, the second angle being closer to perpendicular to the axis of the syringe tip than the first angle.

46. A connector for use with a tapered fitting assembly having at least one attachment member, the connector comprising:
a cooperating tapered fitting; and
a plurality of resilient, extending arms in operative attachment with the cooperating tapered fitting, each of the arms comprising at least one radially inward extending flange, a predetermined level of force being require to cause the radially inward extending flanges to form a cooperating connection with the at least one attachment member of the tapered fitting assembly.

47. The connector of claim 46 wherein a rearward surface of the radially inward extending flanges of the arms is sloped forward to cause the arms to flex radially outward when the connector is moved to contact the radially inward extending flanges of the arms with the attachment member of the tapered fitting assembly which comprises a radially outward extending flange.

48. The connector of claim 47, further comprising a biasing member in operative connection with the cooperating tapered fitting to provide an axial force directed to maintain the tapered fitting assembly and the connector in engagement when the connector is engaged to the tapered fitting assembly.

49. The connector of claim 46, further comprising a retainer to attach a length of tubing thereto by contacting the exterior wall of the tubing.

50. The connector of claim 46, further comprising a valve connected to the port.

51. The connector of claim 50 wherein the valve is a check valve.

52. An injector system for use in a medical injection procedure, the injector system comprising:
a powered injector comprising a drive member;
a syringe comprising an engagement mechanism for removable attachment of the syringe to the injector, a plunger adapted to cooperate with the drive member of the injector, and a syringe tip on a forward end of the syringe, the syringe tip comprising a tapered fitting having a passage therein in fluid communication with an interior of the syringe, the syringe further comprising a first connecting member having at least one attachment member; and
a connector comprising a cooperating tapered fitting, a cooperating attachment member to engage the attachment member of the first connecting member, and at least one port in fluid connection with the cooperating tapered fitting, a predetermined level of force being require to cause the cooperating attachment member to form a connection with the at least one attachment member of the first connecting member.

53. A connector for use with a tapered fitting assembly having at least one attachment member, the connector comprising:
a cooperating tapered fitting;
a cooperating attachment member operable to engage the attachment member of the tapered fitting assembly; and
at least one port in fluid connection with the cooperating tapered fitting; the cooperating attachment member comprising a plurality of extending arms, each of the arms comprising at least one radially inward extending attaching element, the connector further comprising a biasing member to provide axially oriented force directed to retain the tapered fitting assembly and the cooperating tapered fitting of the connector in sealing engagement.

54. The connector of claim 53 wherein the biasing member comprises at least one spring arm in operative connection with the cooperating tapered fitting.

55. The connector of claim 53 wherein the connector is formed from an integral piece of resilient polymeric material.

56. The connector of claim 54 wherein the connector is formed from an integral piece of resilient polymeric material.

57. A method for fabricating a connector for use with a tapered fitting assembly having at least one attachment member, the method comprising the step of forming the connector from an integral piece of polymeric material, the connector being formed to include a cooperating tapered fitting, a cooperating attachment member to engage the attachment member of the tapered fitting assembly, and at least one port in fluid connection with the cooperating tapered fitting; the cooperating attachment member comprising a plurality of extending arms, each of the arms having at least one radially inward extending attaching element, the connector further comprising a biasing member to provide axially oriented force directed to retain the tapered fitting assembly and the cooperating tapered fitting of the connector in sealing engagement.

58. The method of claim 57 wherein the biasing member comprises at least one spring arm attached to the cooperating tapered fitting.

59. A method of connecting a connector comprising a tapered fitting with a tapered fitting assembly comprising a cooperating tapered fitting, the method comprising the steps of forming an axial compressive force directed to maintain connection between the tapered fitting of the connector and the cooperating tapered fitting of the tapered fitting assembly under pressure, and indicating to the user that the axial compressive force has been attained.

60. The method of claim 59, further comprising the step of engaging radially inward extending flanges of extending, resilient arms of the connector with at least one abutment member of the tapered fitting assembly, the radially inward extending flanges being biased radially inward to engage the abutment member of the tapered fitting assembly.

61. An adapter for use in a medical injection procedure in connection with connectors having a tapered fitting, the adapter comprising:
a forward portion comprising a tapered fitting;
a surface surrounding at least a portion of the male tapered fitting, the surface comprising at least one attachment member to connect to a connector comprising a female tapered fitting and a cooperating attachment member to engage the at least one attachment member; and
a rearward portion comprising a rearward fitting in fluid connection with the male tapered fitting.

62. The adapter of claim 61 wherein the forward fitting is a male luer fitting and the rearward fitting is a female luer fitting.

63. The adapter of claim 61 wherein the surface comprises a radially inward threaded portion on an inner surface thereof.

64. The adapter of claim 61 wherein the male tapered fitting, the female tapered fitting and the surface of the adapter are formed integrally.

65. The adapter of claim 64 wherein the adapter is formed of a molded polymeric material.

66. An adapter and connector system for use in a medical injection procedure, the system comprising:
an adapter comprising a forward portion having a male tapered fitting, a surface surrounding at least a portion of the male tapered fitting, the surface comprising at least one attachment member; and a rearward portion comprising a rearward fitting in fluid connection with the male tapered fitting; and
a connector comprising a female tapered fitting to engage the male tapered fitting of the adapter, a cooperating attachment member to engage the at least one attachment member of the adapter, and at least one port in fluid connection with the female tapered fitting.

67. The system of claim 66 wherein the surface comprises a radially inward threaded portion on an inner surface thereof.

68. The system of claim 66 wherein rearward fitting is a female luer fitting and the attachment member of the adapter comprises an abutment surface to form a connection with at least one radially inward extending flange of the cooperating attachment member.

69. The system of claim 66 wherein the attachment member comprises a single flange oriented generally perpendicular to an axis of the adapter.

70. The system of claim 66 wherein the attachment member comprises radially outward extending threading and the cooperating attachment member of the connector comprises a threaded portion to engage the threading of the attachment member.

71. The system of claim 70 wherein the threaded portion of the connector is rotatable relative to the cooperating tapered fitting.

72. A connector for use in a medical procedure to connect to a fitting assembly comprising an interference fitting, comprising: a cooperating interference fitting and an indicator to inform the user that the interference fitting of the fitting assembly and the cooperating interference fitting of the connector have been brought together with an axial compressive force suitable to maintain sealed connection between the tapered fitting of the connector and the cooperating tapered fitting of the tapered fitting assembly under a known pressure.

73. The connector of claim 72 wherein the indicator comprises an attachment member on the connector that comes into connection with a cooperating attachment member on the fitting assembly, the connection resisting axial disconnection of the connector from the fitting assembly.

74. The connector of claim 73 wherein the attachment member on the connector comprises at least one axially extending, flexible arm comprising a radially inward projecting abutment member to connect to the cooperating attachment member.

75. The connector of claim 74 wherein the interference fitting of the fitting assembly is a tapered fitting and the cooperating interference fitting of the connector is a cooperating tapered fitting.

76. The connector of claim 75 wherein the tapered fitting of the fitting assembly is a luer fitting and the tapered fitting of the connector is a cooperating luer fitting.

77. The connector of claim 72 wherein at least one of the interference fitting of the fitting assembly and the cooperating interference fitting of the connector comprises a deformable surface to improve sealed connection therebetween.

78. The connector of claim 72 wherein an axial compressive force is maintained between the interference fitting of the fitting assembly and the cooperating interference fitting of the connector while connected.

79. A connector for use in a medical procedure to connect to a fitting assembly comprising a fitting and an attachment member, comprising: a cooperating fitting comprising an annular deformable seal, a port in fluid connection with the cooperating fitting, and a cooperating attachment member, the cooperating attachment member cooperating with the attachment member to maintain the connector in connection with the fitting assembly so that the deformable seal maintains a sealed connection with the fitting of the fitting assembly.

80. The connector of claim 79 wherein the annular seal in fluid contact with a fluid flowing through the connector and a force with which the annular seal contacts the fitting of the fitting assembly increases with increasing fluid pressure.

81. A connector for connection to a male fitting assembly comprising a surface surrounding a male fitting, the connector comprising a length of deformable conduit, the end of the deformable conduit comprising a radially outward extending flange dimensioned to form a sealed connection with the surrounding surface.

82. The connector of claim 81 wherein the surrounding surface of the male fitting assembly comprises radially inward extending threading, the flange of the deformable conduit abutting the threading to prevent disconnection of the connector from the male fitting assembly when under fluid pressure.

83. The connector of claim 82 wherein the male fitting is a male luer fitting.

84. A connector for connection to a male fitting assembly comprising a male fitting, the connector comprising a length of deformable conduit and a slidable collet surrounding the conduit, the conduit is dimensioned to form a sealed connection between the male fitting and the deformable conduit when the deformable conduit is connected to the male fitting and the collet is slid thereover.

85. The connector of claim 84 wherein the male fitting assembly comprises a generally cylindrical surface surrounding the male fitting, the conduit dimensioned to slide between the deformable conduit and generally cylindrical surface.

86. A connector comprising:
a male fitting assembly comprising a male interference fitting and a generally cylindrical barrel surrounding the male fitting, the barrel having at least one pitched flange positioned adjacent a generally axially oriented slot on the interior wall thereof; and
a female fitting assembly comprising a female interference fitting and at least one radially outward projection dimensioned to pass through the slot of the male fitting assembly and be rotated into abutment with the at least one pitched flange.

87. The connector of claim 86 wherein the male fitting assembly comprises two generally axially oriented slots positioned generally opposite of each other, a first plurality of pitched flanges between the slots on one side of the interior of the barrel and a second plurality of pitched flanges between the slots on another side of the interior of the barrel.

88. The connector of claim 87 wherein the female fitting assembly comprises two radially outward projections, each projection dimensioned to pass through one of the slots of the male fitting assembly and be rotated into abutment with one of the pitched flanges.

89. The connector of claim 88 wherein the female member comprises a stop member to stop rotation of the female fitting assembly after a predetermine amount of rotation.

90. The connector of claim 88 wherein the rotation required to form a sealed connection between the female fitting and the male fitting is no more than 90°.
